(19) United States
(12) Patent Application Publication
(10) Pub. No.: US 2005/0142571 A1
(43) Pub. Date: Jun. 30, 2005

(54) Methods for nucleic acid isolation and kits using solid phase material

(75) Inventors: Ranjani V. Parthasarathy, Woodbury, MN (US); Katya Ericson, Fairburn, GA (US); William Bedingham, Woodbury, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/852,645

(22) Filed: May 24, 2004

Related U.S. Application Data
(60) Provisional application No. 60/532,523, filed on Dec. 24, 2003.

(51) Int. Cl. C12Q 1/68; C07H 21/04; C12N 1/08

(52) U.S. Cl. 435/6; 435/270; 536/25.4

(57) ABSTRACT

The present invention provides methods and kits for isolating nucleic acid from a sample, preferably from a biological sample, using solid phase material.

### CROSS-REFERENCE TO RELATED CASES

The present application claims priority to U.S. Provisional Application Ser. No. 60/532,523, filed on Dec. 24, 2003, which is incorporated herein by reference in its entirety.

### BACKGROUND

The isolation and purification of nucleic acids (DNA and RNA, for example) from complex matrices such as blood, tissue samples, bacterial cell culture media, and forensic samples is an important process in genetic research, nucleic acid probe diagnostics, forensic DNA testing, and other areas that require amplification of the nucleic acids. A variety of methods of preparing nucleic acids for amplification procedures are known in the art; however, each has its limitations.

The most common method for isolating DNA from whole blood involves the isolation of peripheral blood mononuclear cells (PBMC's) using density gradients. While this method works for research applications, it is generally not suitable for use in a conventional integrated, high throughput microfluidic device.

Hypotonic buffers containing a nonionic detergent can be used to lyse red blood cells (RBC's) as well as white blood cells (WBC's) while leaving the nuclei in tact (i.e., unbroken). In another procedure, only RBC's are lysed when whole blood is subjected to freezing and thawing. The in-tact WBC's or their nuclei can be recovered by centrifugation. For lysis of RBC's without destruction of WBC's, one can also use aqueous dilution as a method. Other methods for selective lysis of RBC's include the use of ammonium chloride or quaternary ammonium salts as well as subjecting RBC's to hypotonic shock in the presence of a hypotonic buffer. However, in conventional methods using one of these approaches, substances that inhibit PCR (e.g., inhibitors of enzymes) are coprecipitated with the nuclei and/or nucleic acid. These inhibitors have to be removed prior to analysis in a conventional high throughput microfluidic device.

While treatment such as boiling, hydrolysis with proteinases, exposure to ultrasonic waves, detergents, or strong bases have been used for the extraction of DNA, alkaline extraction is among the simplest of strategies. For example, U.S. Pat. No. 5,620,852 (Lin et al.) describes an efficient extraction of DNA from whole blood performed with alkaline treatment (e.g., NaOH) at room temperature in a time frame as short as 1 minute. However, in order to get clean DNA, removal of hemoglobin as well as plasma proteins is necessary. This has been accomplished by the use of a brief washing step, for example, by suspension of the blood in water followed by centrifugation, discarding of the supernatant, and then extraction of the pellet with NaOH (see, e.g., Biotechniques, Vol. 25, No. 4 (1998) page 588). The large volume of water used to lyse the cells makes the method unsuitable for use in standard microfluidic devices.

U.S. Pat. No. 5,010,183 (Kellogg et al.) describes a centrifugal microfluidics-based platform that uses alkaline lysis for DNA extraction from blood. This method involves mixing a raw sample (e.g., 5 microliters (μL) of whole blood or an E. Coli suspension) with 5 μL of 10 millimolar (mM) NaOH, heating to 95° C. for 1-2 minutes to lyse cells, releasing DNA and denaturing proteins inhibitory to PCR, neutralizing of the lysate by mixing with 5 μL of 16 mM TRIS-HCl (pH 7.5), mixing the neutralized lysate with 8-10 μL of liquid PCR reagents and primers, followed by thermal cycling. Unfortunately, while the reagent volumes are small and suitable for a microfluidic device, downstream processing of DNA in a microfluidic device is challenging.

Another conventional method uses a phenol chloroform extraction. However, this requires the use of toxic and corrosive chemicals and is not easily automated.

Solid phase extraction has also been used for nucleic acid isolation. For example, one method for isolating nucleic acids from a nucleic acid source involves mixing a suspension of silica particles with a buffered chaotropic agent, such as guanidinium thiocyanate, in a reaction vessel followed by addition of the sample. In the presence of the chaotrope, the nucleic acids are adsorbed onto the silica, which is separated from the liquid phase by centrifugation, washed with an alcohol water mix, and finally eluted using a dilute aqueous buffer. Silica solid phase extraction requires the use of the alcohol wash step to remove residual chaotrope without eluting the nucleic acid; however, great care must be taken to remove all traces of the alcohol (by heat evaporation or washing with another very volatile and flammable solvent) in order to prevent inhibition of sensitive enzymes used to amplify or modify the nucleic acid in subsequent steps. The nucleic acid is then eluted with water or an elution buffer. This bind, rinse, and elute procedure is the basis of many commercial kits, such as Qiagen (Valencia, Calif.); however, this procedure is very cumbersome and includes multiple wash steps, making it difficult to adapt to a microfluidic setting.

Ion exchange methods produce high quality nucleic acids. However, ion exchange methods result in the presence of high levels of salts that typically must be removed before the nucleic acids can be further utilized.

International Publication No. WO 01/37291 A1 (MagNA Pure) describes the use of magnetic glass particles and an isolation method in which samples are lysed by incubation with a special buffer containing a chaotropic salt and proteinase K. Glass magnetic particles are added and total nucleic acids contained in the sample are bound to their surface. Unbound substances are removed by several washing steps. Finally, purified total nucleic acid is eluted with a low salt buffer at high temperature.

Yet another conventional method involves applying a biological sample to a hydrophobic organic polymeric solid phase to selectively trap nucleic acid and subsequently remove the trapped nucleic acid with a nonionic surfactant. Another method involves treating a hydrophobic organic polymeric material with a nonionic surfactant, washing the surface, and subsequently contacting the treated solid organic polymeric material with a biological sample to reduce the amount of nucleic acid that binds to the organic polymeric solid phase. Although these solid phase methods are effective methods for isolating nucleic acid from biological samples, other methods are needed, particularly methods that are suitable for use in microfluidic devices.

The discussion of prior publications and other prior knowledge does not constitute an admission that such material was published, known, or part of the common general knowledge.

### SUMMARY

The present invention provides methods for the isolation, and preferably purification and recovery, of nucleic acids.

Nucleic acids isolated according to the invention, will be useful, for example, in assays for detection of the presence of a particular nucleic acid in a sample. Such assays are important in the prediction and diagnosis of disease, forensic medicine, epidemiology, and public health. For example, isolated DNA may be subjected to hybridization and/or amplification to detect the presence of an infectious virus or a mutant gene in an individual, allowing determination of the probability that the individual will suffer from a disease of infectious or genetic origin. The ability to detect an infectious virus or a mutation in one sample among the hundreds or thousands of samples being screened takes on substantial importance in the early diagnosis or epidemiology of an at-risk population for disease, e.g., the early detection of HIV infection, cancer or susceptibility to cancer, or in the screening of newborns for diseases, where early detection may be instrumental in diagnosis and treatment. In addition, the methods of the present invention can also be used in basic research laboratories to isolate nucleic acid from cultured cells or biochemical reactions. The nucleic acid can be used for enzymatic modification such as restriction enzyme digestion, sequencing, and amplification.

The present invention provides methods and kits for isolating nucleic acid from a sample that includes nucleic acid (e.g., DNA, RNA, PNA), which may or may not be included within nuclei-containing cells (e.g., white blood cells). These methods involve ultimately separating nucleic acid from inhibitors, such as heme and degradation products thereof (e.g., iron ions or salts thereof), which are undesirable because they can inhibit amplification reactions (e.g., as are used in PCR reactions).

Certain embodiments of the invention involve retaining inhibitors in or on a solid phase material (i.e., adhering the inhibitors to the material) without retaining a significant amount of nucleic acid. Suitable solid phase materials typically include a solid matrix in any form (e.g., particles, fibrils, a membrane) with capture sites (e.g., chelating functional groups) attached thereto, a coating reagent (preferably, a surfactant) coated on the solid phase material, or both.

In one embodiment, the present invention provides a method of isolating nucleic acid from a sample, the method including: providing a sample including nucleic acid-containing material and inhibitors (typically, contained in cells; such nucleic acid-containing material and cells containing inhibitors may be the same or different); if the sample includes cells containing inhibitors, the method includes optionally contacting the sample with a first lysing reagent under conditions effective to break cell membranes and release inhibitors and form a lysed sample including nucleic acid-containing material and inhibitors; forming a concentrated region of the sample (typically, lysed sample); wherein the concentrated region of the sample (typically, lysed sample) includes nucleic acid-containing material and inhibitors; substantially separating the concentrated region from a less concentrated region of the sample (typically, lysed sample); contacting the separated concentrated region of the sample (typically, lysed sample) with a solid phase material to preferentially adhere at least a portion of the inhibitors (i.e., at least a portion of at least one inhibitor) to the solid phase material, wherein the solid phase material includes a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof; optionally further lysing the nucleic acid-containing material to release nucleic acid before, simultaneous with, or after contacting the separated concentrated region of the sample with the solid phase material; and separating at least a portion of the nucleic acid-containing material and/or nucleic acid from the solid phase material having at least a portion of the inhibitors adhered thereto.

In another embodiment, the present invention provides a method of isolating nucleic acid from a sample, the method including: providing a sample including cells containing inhibitors and cells containing nuclei (such cells containing inhibitors and cells containing nuclei may be the same or different); contacting the biological sample with a nonionic surfactant under conditions effective to break cell membranes and release nuclei and inhibitors and form a lysed sample; forming a concentrated region of the lysed sample; wherein the concentrated region of the sample includes nuclei and inhibitors; substantially separating the concentrated region of the lysed sample from the less concentrated region of the sample; contacting the separated concentrated region of the lysed sample with a solid phase material to preferentially adhere at least a portion of the inhibitors to the solid phase material, wherein the solid phase material includes a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof; optionally further lysing the nuclei to release nucleic acid before, simultaneous with, or after contacting the separated concentrated region of the sample with the solid phase material; and separating at least a portion of the nucleic acid-containing and/or nucleic acid from the solid phase material having at least a portion of the inhibitors adhered thereto.

In another embodiment, the present invention provides a method of isolating nucleic acid from a sample, the method including: providing a microfluidic device including a loading chamber, a valved process chamber, and a separation chamber including a solid phase material; providing a sample including nucleic acid-containing material and inhibitors (typically, contained in cells; such nucleic acid-containing material and cells containing inhibitors may be the same or different); placing the sample in the loading chamber; if the sample includes cells containing inhibitors, optionally contacting the sample with a first lysing reagent under conditions effective to break cell membranes and release inhibitors and form a lysed sample including nucleic acid-containing material and inhibitors; transferring the sample (typically, lysed sample) to a valved process chamber; forming a concentrated region of the sample (typically, lysed sample) in the valved process chamber, wherein the concentrated region of the sample (typically, lysed sample) includes nucleic acid-containing material and inhibitors; substantially separating the concentrated region from a less concentrated region of the sample (typically, lysed sample); transferring the separated concentrated region of the sample (typically, lysed sample) to the separation chamber for contact with the solid phase material to preferentially adhere at least a portion of the inhibitors to the solid phase material; wherein the solid phase material includes capture sites (e.g., chelating functional groups), a coating reagent coated on the solid phase material, or both; wherein the coating reagent is selected from the group consisting of a surfactant, a strong base, a polyelectrolyte, a selectively permeable polymeric barrier, and combinations thereof; optionally further lysing the nucleic acid-containing material to release nucleic acid before, simultaneous with, or after contacting the separated concentrated region of the sample with the solid phase material; and separating at least a portion of the nucleic acid-containing material and/or nucleic acid from the solid phase material having at least a portion of the inhibitors adhered thereto.

In another embodiment, the present invention provides a method of isolating nucleic acid from a sample, the method including: providing a microfluidic device including a loading chamber, a valved process chamber, and a separation chamber including a solid phase material; providing a sample including cells containing inhibitors and cells containing nuclei (such cells containing inhibitors and cells containing nuclei may be the same or different); placing the sample in the loading chamber; contacting the sample with a nonionic surfactant under conditions effective to break cell membranes and release nuclei and inhibitors to form a lysed sample; transferring the lysed sample to a valved process chamber; forming a concentrated region of the lysed sample in the valved process chamber, wherein the concentrated region of the lysed sample includes nuclei and inhibitors; substantially separating the concentrated region from a less concentrated region of the lysed sample; transferring the separated concentrated region of the lysed sample to the separation chamber for contact with the solid phase material to preferentially adhere at least a portion of the inhibitors to the solid phase material; wherein the solid phase material includes capture sites (e.g., chelating functional groups), a coating reagent coated on the solid phase material, or both; wherein the coating reagent is selected from the group consisting of a surfactant, a strong base, a polyelectrolyte, a selectively permeable polymeric barrier, and combinations thereof; optionally funrther lysing the nuclei to release nucleic acid before, simultaneous with, or after contacting the separated concentrated region of the sample with the solid phase material; and separating at least a portion of the nuclei and/or nucleic acid from the solid phase material having at least a portion of the inhibitors adhered thereto.

In another embodiment, the present invention provides a method of isolating nucleic acid from a sample, the method including: providing a sample including nucleic acid and inhibitors; contacting the sample with a solid phase material to preferentially adhere at least a portion of the inhibitors to the solid phase material, wherein the solid phase material includes a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof; optionally lysing nucleic acid-containing material, if present, to release nucleic acid before, simultaneous with, or after contacting the lysed sample with the solid phase material; and separating at least a portion of the nucleic acid-containing material and/or nucleic acid from the solid phase material having at least a portion of the inhibitors adhered thereto by eluting with an eluting reagent, with the proviso that the eluting reagent is not a nonionic surfactant.

In another embodiment, the present invention provides a method of isolating nucleic acid from a sample, the method including: providing a sample including nucleic acid-containing material (e.g., nuclei) and inhibitors (typically, contained in cells; such nucleic acid-containing material and cells containing inhibitors may be the same or different); if the sample includes cells containing inhibitors, optionally contacting the sample with a first lysing reagent under conditions effective to break cell membranes and release inhibitors and form a lysed sample including nucleic acid-containing material and inhibitors; contacting the sample (typically, lysed sample) with a solid phase material to preferentially adhere at least a portion of the inhibitors to the solid phase material, wherein the solid phase material includes a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof (preferably, a surfactant); optionally further lysing the nucleic acid-containing material to release nucleic acid before, simultaneous with, or after contacting the lysed sample with the solid phase material; and separating at least a portion of the nucleic acid-containing material and/or nucleic acid from the solid phase material having at least a portion of the inhibitors adhered thereto by eluting with an eluting reagent, with the proviso that the eluting reagent is not a nonionic surfactant.

In another embodiment, the present invention provides a method of isolating nucleic acid from a sample, the method including: providing a sample including nucleic acid-containing material (e.g., nuclei) and cells containing inhibitors (which may be the same or different); contacting the sample with a first lysing reagent under conditions effective to break cell membranes and release inhibitors and form a lysed sample including nucleic acid-containing material and inhibitors; contacting the lysed sample with a solid phase material to preferentially adhere at least a portion of the inhibitors to the solid phase material, wherein the solid phase material includes a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof (preferably, a surfactant); separating at least a portion of the nucleic acid-containing material from the solid phase material having at least a portion of the inhibitors adhered thereto; and after separating the nucleic acid-containing material, further lysing the nucleic acid-containing material to release nucleic acid.

In another embodiment, the present invention provides a method of isolating nucleic acid from a sample, the method including: providing a microfluidic device including a loading chamber and a separation chamber including a solid phase material; providing a sample including nucleic acid-containing material and inhibitors (typically, contained in cells; such nucleic acid-containing material and cells containing inhibitors may be the same or different); placing the sample in the loading chamber; if the sample includes cells containing inhibitors, optionally contacting the sample with a first lysing reagent under conditions effective to break cell membranes and release inhibitors and form a lysed sample including nucleic acid-containing material and inhibitors; transferring the sample (typically, lysed sample) to the separation chamber to contact the solid phase material and to preferentially adhere at least a portion of the inhibitors to the solid phase material; optionally further lysing the nucleic acid-containing material to release nucleic acid before, simultaneous with, or after contacting the sample with the solid phase material; wherein the solid phase material includes capture sites (e.g., chelating functional groups), a coating reagent coated on the solid phase material, or both; wherein the coating reagent is selected from the group consisting of a surfactant, a strong base, a polyelectrolyte, a selectively permeable polymeric barrier, and combinations thereof; and separating at least a portion of the nucleic acid-containing material and/or nucleic acid from the solid phase material having at least a portion of the inhibitors adhered thereto by eluting with an eluting reagent, with the proviso that the eluting reagent is not a nonionic surfactant.

In another embodiment, the present invention provides a method of isolating nucleic acid from a sample, the method including: providing a microfluidic device including a loading chamber and a separation chamber including a solid phase material; providing a sample including nucleic acid-containing material and cells containing inhibitors (which may be the same or different); placing the sample in the loading chamber; contacting the sample with a first lysing reagent under conditions effective to break cell membranes and release inhibitors and form a lysed sample including nucleic acid-containing material and inhibitors; transferring the lysed sample to the separation chamber to contact the solid phase material and to preferentially adhere at least a portion of the inhibitors to the solid phase material; wherein the solid phase material includes capture sites (e.g., chelating functional groups), a coating reagent coated on the solid phase material, or both; wherein the coating reagent is selected from the group consisting of a surfactant, a strong base, a polyelectrolyte, a selectively permeable polymeric barrier, and combinations thereof; and separating at least a portion of the nucleic acid-containing material from the solid phase material having at least a portion of the inhibitors adhered thereto; and after separating the nucleic acid-containing material, further lysing the nucleic acid-containing material to release nucleic acid.

The present invention also provides kits for carrying out the various methods of the present invention.

### Definitions

“Nucleic acid” shall have the meaning known in the art and refers to DNA (e.g., genomic DNA, cDNA, or plasmid DNA), RNA (e.g., mRNA, tRNA, or rRNA), and PNA. It can be in a wide variety of forms, including, without limitation, double-stranded or single-stranded configurations, circular form, plasmids, relatively short oligonucleotides, peptide nucleic acids also called PNA's (as described in Nielsen et al., Chem. Soc. Rev., 26, 73-78 (1997)), and the like. The nucleic acid can be genomic DNA, which can include an entire chromosome or a portion of a chromosome. The DNA can include coding (e.g., for coding mRNA, tRNA, and/or rRNA) and/or noncoding sequences (e.g., centromeres, telomeres, intergenic regions, introns, transposons, and/or microsatellite sequences). The nucleic acid can include any of the naturally occurring nucleotides as well as artificial or chemically modified nucleotides, mutated nucleotides, etc. The nucleic acid can include a non-nucleic acid component, e.g., peptides (as in PNA's), labels (radioactive isotopes or fluorescent markers), and the like.

“Nucleic acid-containing material” refers to a source of nucleic acid such as a cell (e.g., white blood cell, enucleated red blood cell), a nuclei, or a virus, or any other composition that houses a structure that includes nucleic acid (e.g., plasmid, cosmid, or viroid, archeobacteriae). The cells can be prokaryotic (e.g., gram positive or gram negative bacteria) or eukaryotic (e.g., blood cell or tissue cell). If the nucleic acid-containing material is a virus, it can include an RNA or a DNA genome; it can be virulent, attenuated, or noninfectious; and it can infect prokaryotic or eukaryotic cells. The nucleic acid-containing material can be naturally occurring, artificially modified, or artificially created.

“Isolated” refers to nucleic acid (or nucleic acid-containing material) that has been separated from at least a portion of the inhibitors (i.e., at least a portion of at least one inhibitor) in a sample. This includes separating desired nucleic acid from other materials, e.g., cellular components such as proteins, lipids, salts, and other inhibitors. More preferably, the isolated nucleic acid is substantially purified. “Substantially purified” refers to isolating nucleic acid of at least 3 picogram per microliter (pg/μL), preferably at least 2 nanogram/microliter (ng/μL), and more preferably at least 15 ng/μL, while reducing the inhibitor amount from the original sample by at least 20%, preferably by at least 80% and more preferably by at least 99%. The contaminants are typically cellular components and nuclear components such as heme and related products (hemin, hematin) and metal ions, proteins, lipids, salts, etc., other than the solvent in the sample. Thus, the term “substantially purified” generally refers to separation of a majority of inhibitors (e.g., heme and it degradation products) from the sample, so that compounds capable of interfering with the subsequent use of the isolated nucleic acid are at least partially removed.

“Adheres to” or “adherence” or “binding” refer to reversible retention via a wide variety of mechanisms, including weak forces such as Van der Waals interactions, electrostatic interactions, affinity binding, or physical trapping. The use of this term does not imply a mechanism of action, and includes adsorptive and absorptive mechanisms.

“Solid phase material” refers to an inorganic and/or organic material, preferably a polymer made of repeating units, which may be the same or different, of organic and/or inorganic compounds of natural and/or synthetic origin. This includes homopolymers and heteropolymers (e.g., copolymers, terpolymers, tetrapolymers, etc., which may be random or block, for example). This term includes fibrous or particulate forms of a material, which can be readily prepared by methods well-known in the art. Such materials typically form a porous matrix, although for certain embodiments, the solid phase also refers to a solid surface, such as a nonporous sheet of polymeric material.

The solid phase material may include capture sites. “Capture sites” refer to sites on the solid phase material to which a material adheres. Typically, the capture sites include functional groups or molecules that are either covalently attached or otherwise attached (e.g., hydrophobically attached) to the solid phase material.

The phrase “coating reagent coated on the solid phase material” refers to a material coated on at least a portion of the solid phase material, e.g., on at least a portion of the fibril matrix and/or sorptive particles.

“Surfactant” refers to a substance that lowers the surface or interfacial tension of the medium in which it is dissolved.

“Strong base” refers to a base that is completely dissociated in water, e.g., NaOH.

“Polyelectrolyte” refers to an electrolyte that is a charged polymer, typically of relatively high molecular weight, e.g., polystyrene sulfonic acid.

“Selectively permeable polymeric barrier” refers to a polymeric barrier that allows for selective transport of a fluid based on size and charge.

“Concentrated region” refers to a region of a sample that has a higher concentration of nucleic acid-containing material, nuclei, and/or nucleic acid, which can be in a pellet form, relative to the less concentrated region.

“Substantially separating” as used herein, particularly in the context of separating a concentrated region of a sample from a less concentrated region of a sample, means removing at least 40% of the total amount of nucleic acid (whether it be free, within nuclei, or within other nucleic acid-containing material) in less than 25% of the total volume of the sample. Preferably, at least 75% of the total amount of nucleic acid in less than 10% of the total volume of sample is separated from the remainder of the sample. More preferably, at least 95% of the total amount of nucleic acid in less than 5% of the total volume of sample is separated from the remainder of the sample.

“Inibitors” refer to inhibitors of enzymes used in amplification reactions, for example. Examples of such inhibitors typically include iron ions or salts thereof (e.g., Fe²⁺ or salts thereof) and other metal salts (e.g., alkali metal ions, transition metal ions). Other inhibitors can include proteins, peptides, lipids, carbohydrates, heme and its degradation products, urea, bile acids, humic acids, polysaccharides, cell membranes, and cytosolic components. The major inhibitors in human blood for PCR are hemoglobin, lactoferrin, and IgG, which are present in erythrocytes, leukocytes, and plasma, respectively. The methods of the present invention separate at least a portion of the inhibitors (i.e., at least a portion of at least one type of inhibitor) from nucleic acid-containing material. As discussed herein, cells containing inhibitors can be the same as the cells containing nuclei or other nucleic acid-containing material. Inhibitors can be contained in cells or be extracellular. Extracellular inhibitors include all inhibitors not contained within cells, which includes those inhibitors present in serum or viruses, for example.

“Preferentially adhere at least a portion of the inhibitors to the solid phase material” means that one or more types of inhibitors will adhere to the solid phase material to a greater extent than nucleic acid-containing material (e.g., nuclei) and/or nucleic acid, and typically without adhering a substantial portion of the nucleic acid-containing material and/or nuclei to the solid phase material.

“Microfluidic” refers to a device with one or more fluid passages, chambers, or conduits that have at least one internal cross-sectional dimension, e.g., depth, width, length, diameter, etc., that is less than 500 μm, and typically between 0.1 μm and 500 μm. In the devices used in the present invention, the microscale channels or chambers preferably have at least one cross-sectional dimension between 0.1 μm and 200 μm, more preferably between 0.1 μm and 100 μm, and often between 1 μm and 20 μm. Typically, a microfluidic device includes a plurality of chambers (process chambers, separation chambers, mixing chambers, waste chambers, diluting reagent chambers, amplification reaction chambers, loading chambers, and the like), each of the chambers defining a volume for containing a sample; and at least one distribution channel connecting the plurality of chambers of the array; wherein at least one of the chambers within the array can include a solid phase material (thereby often being referred to as a separation chamber) and/or at least one of the process chambers within the array can include a lysing reagent (thereby often being referred to as a mixing chamber), for example.

The terms “comprises” and variations thereof do not have a limiting meaning where these terms appear in the description and claims.

As used herein, “a,” “an,” “the,” “at least one,” and “one or more” are used interchangeably and mean one or more.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list. Furthermore, various embodiments are described in which the various elements of each embodiment could be used in other embodiments, even though not specifically described.

### BRIEF DESCRIPTION OF THE DRAWINGS

FIGS. 1-2 are representations of microfluidic devices used in certain methods of the present invention.

### DETAILED DESCRIPTION OF ILLUSTRATIVE EMBODIMENTS

The present invention provides various methods and kits for isolating nucleic acid from a sample, typically a biological sample, preferably in a substantially purified form. The present invention provides methods and kits for isolating nucleic acid from a sample that includes nucleic acid (e.g., DNA, RNA, PNA), which may or may not be included within nuclei-containing cells (e.g., white blood cells).

It should be understood that although the methods are directed to isolating nucleic acid from a sample, the methods do not necessarily remove the nucleic acid from the nucleic acid-containing material (e.g., nuclei). That is, further steps may be required to further separate the nucleic acid from the nuclei, for example.

The methods of the present invention involve ultimately separating nucleic acid from inhibitors, such as heme and degradation products thereof (e.g., iron salts), which are undesirable because they can inhibit amplification reactions (e.g., as are used in PCR reactions). More specifically, the methods of the present invention involve separating at least a portion of the nucleic acid in a sample from at least a portion of at least one type of inhibitor. Preferred methods involve removing substantially all the inhibitors in a sample containing nucleic acid such that the nucleic acid is substantially pure. For example, the final concentration of iron-containing inhibitors is no greater than about 0.8 micromolar (μM), which is the current level tolerated in conventional PCR systems.

In order to get clean DNA from whole blood, removal of hemoglobin as well as plasma proteins is typically desired. When red blood cells are lysed, heme and related compounds are released that inhibit Taq Polymerase. The normal hemoglobin concentration in whole blood is 15 grams (g) per 100 milliliters (mL) based on which the concentration of heme in hemolysed whole blood is around 10 millimolar (mM). For PCR to work out satisfactorily, the concentration of heme should be reduced to the micromolar (μM) level. This can be achieved by dilution or by removal of inhibitors using a material that binds inhibitors, for example.

Typically, a sample containing nucleic acid is processed in a flow-through receptacle, although this receptacle is not a necessary requirement of the present invention. Preferably, for certain methods of the present invention, the processing equipment is in a microfluidic format.

### Samples

The methods of the present invention can be used to isolate nucleic acids from a wide variety of samples, particularly biological samples, such as body fluids (e.g., whole blood, blood serum, urine, saliva, cerebral spinal fluid, semen, or synovial lymphatic fluid), various tissues (e.g., skin, hair, fur, feces, tumors, or organs such as liver or spleen), cell cultures or cell culture supernatants, etc. The sample can be a food sample, a beverage sample, a fermentation broth, a clinical sample used to diagnose, treat, monitor, or cure a disease or disorder, a forensic sample, an agricultural sample (e.g., from a plant or animal), or an environmental sample (e.g., soil, dirt, or garbage).

Biological samples are those of biological or biochemical origin. Those suitable for use in the methods of the present invention can be derived from mammalian, plant, bacterial, or yeast sources. The biological sample can be in the form of single cells or in the form of a tissue. Cells or tissue can be derived from in vitro culture. Significantly, certain embodiments of the invention use whole blood without any preprocessing (e.g., lysing, filtering, etc.) as the sample of interest.

For certain embodiments, a sample such as whole blood can be preprocessed by centrifuging and the white blood cells (i.e., the buffy coat) separated from the blood and used as the sample in the methods of the invention.

For certain embodiments, a sample can be subjected to ultracentrifligation to concentrate the sample prior to subjecting it to a process of the present invention.

The sample can be a solid sample (e.g., solid tissue) that is dissolved or dispersed in water or an organic medium, or from which the nucleic acid has been extracted into water or an organic medium. For example, the sample can be an organ homogenate (e.g., liver, spleen). Thus, the sample can include previously extracted nucleic acid (particularly if it is a solid sample).

The type of sample is not a limitation of the present invention. Typically, however, the sample will include nucleic acid-containing material and inhibitors from which the nucleic acid needs to be separated. In this context, nucleic acid-containing material refers to cells (e.g., white blood cell, bacterial cells), nuclei, viruses, or any other composition that houses a structure that includes nucleic acid (e.g., plasmid, cosmid, or viroid, archeobacteriae). In certain preferred embodiments of such methods, the nucleic acid-containing material includes nuclei. In certain embodiments, such nuclei are in tact (i.e., substantially unlysed) when they contact the solid phase material described herein.

In certain embodiments, the sample may be partially lysed (e.g., pre-lysed to release inhibitors), in which case lysing may be required in the process of the present invention, or fully lysed. Thus, a sample can include free (e.g., not within cells) nucleic acid and free (e.g., not within cells) inhibitors.

The isolated (i.e., separated from inhibitors) nucleic acid can be used, preferably without further purification or washing, for a wide variety of applications (e.g., amplification, sequencing, labeling, annealing, restriction digest, ligation, reverse transcriptase, hybridization, Southern blot, Northern blot, etc.). In particularly, it can be used for determining a subject's genome. It can be used for the diagnosis of the presence of a microorganism (e.g., bacteria, virus) in a sample, and subsequently can be used for monitoring and/or remedying the damage caused by the microorganism to the source of the sample. The methods, materials, systems, and kits of the present invention are especially well-suited for preparing nucleic acid extracts for use in amplification techniques (e.g., PCR, LCR, MASBA, SDA, and bDNA) used in high throughput or automated processes, particularly microfluidic systems. Thus, for certain embodiments of the present invention, the isolated nucleic acid is transferred to an amplification reaction chamber (such as a PCR sample chamber in a microfluidic device).

The nucleic acids may be isolated (i.e., separated from inhibitors) according to the invention from an impure, partially pure, or a pure sample. The purity of the original sample is not critical, as nucleic acid may be isolated from even grossly impure samples. For example, nucleic acid may be obtained from an impure sample of a biological fluid such as blood, saliva, or tissue. If an original sample of higher purity is desired, the sample may be treated according to any conventional means known to those of skill in the art prior to undergoing the methods of the present invention. For example, the sample may be processed so as to remove certain impurities such as insoluble materials prior to subjecting the sample to a method of the present invention.

The nucleic acid isolated as described herein may be of any molecular weight and in single-stranded form, double-stranded form, circular, plasmid, etc. Various types of nucleic acid can be separated from each other (e.g., RNA from DNA, or double-stranded DNA from single-stranded DNA). For example, small oligonucleotides or nucleic acid molecules of about 10 to about 50 bases in length, much longer molecules of about I1000 bases to about 10,000 bases in length, and even high molecular weight nucleic acids of about 50 kb to about 500 kb can be isolated using the methods of the present invention. In some aspects, a nucleic acid isolated according to the invention may preferably be in the range of about 10 bases to about 100 kilobases.

The nucleic acid-containing sample may be in a wide variety of volumes. For example, the applied volume may be as large as 1 liter or as small as 1 μL, or even less. The sample size typically varies depending on the equipment used to carry out the method. For a microfluidic format, typically very small volumes, e.g., 10 μL (and preferably, no greater than 100 μL) are preferred. It should be understood that larger samples can be used if preprocessed, such as by concentrating.

For low copy number genes, one typically would need a larger sample size to ensure that the sequence of interest is present in the sample. Larger sample sizes, however, have a greater amount of inhibitors and do not typically lend themselves to a microfluidic format. Thus, for a low copy number situation, it may be necessary to use a 100 μL or higher volume in order to get a reproducible result; however, the number of samples processed per microfluidic device may be reduced due to the higher sample volume.

In the methods of the present invention, a centrifugation step to concentrate nucleic acid-containing material is usefuil for low copy number samples. However, while the nucleic acid concentration is increased substantially at the bottom of the process chamber, the inhibitor concentration is still high. While most of the inhibitors, the proteins in the serum and the broken RBC's (e.g., heme and heme-related products) are removed in the supernatant, the nucleic acid-containing concentrated region of the sample still has a significant amount of inhibitor present; however, the ratio of nucleic acid to that of the inhibitor is very high, resulting in an enriched sample with respect to nucleic acid. This concentrated region of the sample can then be contacted with a solid phase material, as described herein, to remove residual inhibitors.

For high copy number genes, a sample size as small as 2 μL can be used, but reproducibility is better with larger volumes (e.g., 20 μL). In the case of smaller volumes, higher throughputs (i.e., number of samples processed per microfluidic device) can be obtained. In the case of larger volumes (e.g., 20 μL), it may not be necessary to go through a pre-spin step for concentration of nucleic acid-containing cells.

For certain embodiments, the nucleic acid-containing sample applied to the solid phase material may be any amount, that amount being determined by the amount of the solid phase material. Preferably, the amount of nucleic acid in a sample applied to the solid phase material is less than the dried weight of the solid phase material, typically about 1/10,000 to about 1/100 (weight nucleic acid/solid phase). The amount of nucleic acid in a sample applied to the solid phase material may be as much as 100 grams or as little as 1 picogram, for example.

The desired nucleic acid isolated from the methods of the present invention is preferably in an amount of at least 20%, more preferably in an amount of at least 30%, more preferably at least 70%, and most preferably at least 90%, of the amount of total nucleic acid in the originally applied sample. Thus, certain preferred methods of the present invention provide for high recovery of the desired nucleic acid from a sample. Furthermore, exceedingly small amounts of nucleic acid molecules may be quantitatively recovered according to the invention. The recovery or yield is mainly dependent on the quality of the sample rather than the procedure itself. Because certain embodiments of the invention provide a nucleic acid preparation that does not require concentration from a large volume, the invention avoids risk of loss of the nucleic acid.

Having too much DNA in a PCR sample can be detrimental to amplification of DNA as there are a lot of misprimed sites. This results in a large number of linearly or exponentially amplified non-target sequences. Since the specificity of the amplification is lost as the amount of non-target DNA is increased, the exponential accumulation of the target sequence of interest does not occur to any significant degree. Thus, it is desirable to control the amount of DNA that goes into each PCR sample. The DNA amount is typically not more than 1 microgram/reaction, typically at least 1 picogram/reaction. The typical final DNA concentration in a PCR mixture ranges from 0.15 nanogram/microliter to 1.5 nanograms/microliter. In the case of a microfluidic device, a sample can be split after clean-up, prior to PCR, such that each sample has the right amount of DNA. Alternatively, a sample can be diluted sufficiently in a sample processing device (particularly, a microfluidic device) that includes a variable valved process chamber, described in greater detail below, so that the right amount of DNA is present in each PCR mixture. In a diagnostic setting, since the amount of white blood cells can vary significantly, it is hard to apriori predict the amount of DNA that will be isolated. However, a useful range is 3 micrograms (μg) to 12 μg of DNA per 200 μL of blood. For buffy coats, 25 μg to 50 μg per 200 μL of buffy coat is a useful range.

### Lysing Reagents and Conditions

For certain embodiments of the invention, at some point during the process, cells within the sample, particularly nucleic acid-containing cells (e.g., white blood cells, bacterial cells, viral cells) are lysed to release the contents of the cells and form a sample (i.e., a lysate). Lysis herein is the physical disruption of the membranes of the cells, referring to the outer cell membrane and, when present, the nuclear membrane. This can be done using standard techniques, such as by hydrolyzing with proteinases followed by heat inactivation of proteinases, treating with surfactants (e.g., nonionic surfactants or sodium dodecyl sulfate), guanidinium salts, or strong bases (e.g., NaOH), disrupting physically (e.g., with ultrasonic waves), boiling, or heating/cooling (e.g., heating to at least 55° C. (typically to 95° C.) and cooling to room temperature or below (typically to 8° C.)), which can include a freezing/thawing process. Typically, if a lysing reagent is used, it is in aqueous media, although organic solvents can be used, if desired.

Lysing of red blood cells (RBC's) without the destruction of white blood cells (WBC's) in whole blood can occur to release inhibitors through the use of water (i.e., aqueous dilution) as the lysing agent (i.e., lysing reagent). Alternatively, ammonium chloride or quaternary ammonium salts can also be used to break RBC's. The RBC's can also be lysed by hypotonic shock with the use of a hypotonic buffer. The in-tact WBC's or their nuclei can be recovered by centrifugation, for example.

Typically, a stronger lysing reagent, such as a surfactant, can be used to lyse RBC's as well as nucleic acid-containing cells (e.g., white blood cells (WBC's), bacterial cells, viral cells) to release inhibitors, nuclei, and/or nucleic acid. For example, a nonionic surfactant can be used to lyse RBC's as well as WBC's while leaving the nuclei in tact. Nonionic surfactants, cationic surfactants, anionic surfactants, and zwitterionic surfactants can be used to lyse cells. Particularly useful are nonionic surfactants. Combinations of surfactants can be used if desired. A nonionic surfactant such as TRITON X-100 can be added to a TRIS buffer containing sucrose and magnesium salts for isolation of nuclei.

The amount of surfactant used for lysing is sufficiently high to effectively lyse the sample, yet sufficiently low to avoid precipitation, for example. The concentration of surfactant used in lysing procedures is typically at least 0.1 wt-%, based on the total weight of the sample. The concentration of surfactant used in lysing procedures is typically no greater than 4.0 wt-%, and preferably, no greater than 1.0 wt-%, based on the total weight of the sample. The concentration is usually optimized in order to obtain complete lysis in the shortest possible time with the resulting mixture being PCR compatible. In fact, the nucleic acid in the formulation added to the PCR cocktail should allow for little or no inhibition of real-time PCR.

If desired, a buffer can be used in admixture with the surfactant. Typically, such buffers provide the sample with a pH of at least 7, and typically no more than 9.

Typically, an even stronger lysing reagent, such as a strong base, can be used to lyse any nuclei contained in the nucleic acid-containing cells (as in white blood cells) to release nucleic acid. For example, the method described in U.S. Pat. No. 5,620,852 (Lin et al.), which involves extraction of DNA from whole blood with alkaline treatment (e.g., NaOH) at room temperature in a time frame as short as 1 minute, can be adapted to certain methods of the present invention. Generally, a wide variety of strong bases can be used to create an effective pH (e.g., 8-13, preferably 13) in an alkaline lysis procedure. The strong base is typically a hydroxide such as NaOH, LiOH, KOH; hydroxides with quaternary nitrogen-containing cations (e.g., quaternary ammonium) as well as bases such as tertiary, secondary or primary amines. Typically, the concentration of the strong base is at least 0.01 Normal (N), and typically, no more than 1 N. Typically, the mixture can then be neutralized, particularly if the nucleic acid is subjected to PCR. In another procedure, heating can be used subsequent to lysing with base to further denature proteins followed by neutralizing the sample.

One can also use Proteinase K with heat followed by heat inactivation of proteinase K at higher temperatures for isolation of nucleic acids from the nuclei or WBC.

One can also use a commercially available lysing agent and neutralization agent such as in Sigma's Extract-N-Amp Blood PCR kit scaled down to microfluidic dimensions. Stonger lysing solutions such as POWERLYSE from GenPoint (Oslo, Norway) for lysing difficult bacteria such as Staphylococcus, Streptococcus, etc., can be used to advantage in certain methods of the present invention.

In another procedure, a boiling method can be used to lyse cells and nuclei, release DNA, and precipitate hemoglobin simultaneously. The DNA in the supernatant can be used directly for PCR without a concentration step, making this procedure useful for low copy number samples.

For infectious diseases, it may be necessary to analyze bacterial or viruses from whole blood. For example, in the case of bacteria, white blood cells may be present in conjunction with bacterial cells. In methods that use a microfluidic device, it would be possible to lyse red blood cells to release inhibitors, and then separate out bacterial cells and white blood cells by centrifugation, for example, prior to further lysing. This concentrated slug of nucleic acid-containing cells (bacterial and white blood cells/nuclei) can be moved further into a chamber containing the solid phase material for removal of inhibitors. Then, the bacterial cells, for example, can be lysed.

Bacterial cell lysis, depending on the type, may be accomplished using heat. Alternatively, bacterial cell lysis can occur using enzymatic methods (e.g., lysozyme, mutanolysin) or chemical methods. The bacterial cells are preferably lysed by alkaline lysis.

The use of bacteria for propagation of plasmids is common in the study of genomics, analytic molecular biology, preparatory molecular biology, etc. In the case of the bacterium containing plasmid, genetic material from both the bacterium and the plasmid are present. A clean-up procedure to separate cellular proteins and cellular fragments from genomic DNA can be carried out using a method of the present invention. The supernatant thus obtained, which contains the plasmid DNA, is called the “cleared lysate.” The cleared lysate can be further purified using a variety of means, such as anion-exchange chromatography, gel filtration, or precipitation with alcohol.

In a specific example of a protocol for bacterial cultures, which can be incorporated into a microfluidic device, an E. Coli cell culture is centrifuged and resuspended in TE buffer (10 mM TRIS, 1 mM EDTA, pH 7.5) and lysed by the addition of 0.1 M NaOH/1% SDS (sodium dodecyl sulfate). The cell lysis is stopped by the addition of 1 volume of 3 M (three molar) potassium acetate (pH 4.8) and the supernatant centrifuged. The cell lysate is further purified to get clean plasmid DNA.

Plasma and serum represent the majority of specimens submitted for molecular testing that include viruses. After fractionation of whole blood, plasma or serum samples can be used for the extraction of viruses (i.e., viral particles). For example, to isolate DNA from viruses, it is possible in the microfluidic case, to first separate out the serum by spinning blood. By the use of the variable valve, which is described in greater detail below, the serum alone can be emptied into another chamber. The serum can then be centrifuged to concentrate the virus or can be used directly in subsequent lysis steps after removal of the inhibitors using a solid phase material, for example, as described herein. The solid phase material could absorb the solution such that the virus particles do not go through the material. The virus particles can then be eluted out in a small elution volume. The virus can be lysed by heat or by enzymatic or chemical means, for example, by the use of surfactants, and used for downstream applications, such as PCR or real-time PCR. In cases where viral RNA is required, it may be necessary to have an RNAse inhibitor added to the solution to prevent degradation of RNA.

### Solid Phase Material

For certain embodiments of the invention, it has been found that inhibitors will adhere to solid phase (preferably polymeric) materials that include a solid matrix in any form (e.g., particles, fibrils, a membrane), preferably with capture sites (e.g., chelating functional groups) attached thereto, a coating reagent (preferably, surfactant) coated on the solid phase material (i.e., at least a portion thereof), or both. The coating reagent can be a cationic, anionic, nonionic, or zwitterionic surfactant. Alternatively, the coating reagent can be a polyelectrolyte, a strong base, or a selectively permeable polymeric barrier. Various combinations of coating reagents can be used if desired.

The solid phase material useful in the methods of the present invention may include a wide variety of organic and/or inorganic materials that retain inhibitors such as heme and heme degradation products, particularly iron ions, for example. Such materials are functionalized with capture sites (preferably, chelating groups), coated with one or more coating reagents (e.g., surfactants, polyelectrolytes, or strong bases), or both. Typically, the solid phase material includes an organic polymeric matrix.

Generally suitable materials are chemically inert, physically and chemically stable, and compatible with a variety of biological samples. Examples of solid phase materials include silica, zirconia, alumina beads, metal colloids such as gold, gold coated sheets that have been functionalized through mercapto chemistry, for example, to generate capture sites. Examples of suitable polymers include for example, polyolefins and fluorinated polymers. The solid phase material is typically washed to remove salts and other contaminants prior to use. It can either be stored dry or in aqueous suspension ready for use. The solid phase material is preferably used in a flow-through receptacle, for example, such as a pipet, syringe, or larger column, microtiter plate, or microfluidic device, although suspension methods that do not involve such receptacles could also be used.

The solid phase material useful in the methods of the present invention can include a wide variety of materials in a wide variety of forms. For example, it can be in the form of particles or beads, which may be loose or immobilized, fibers, foams, fiits, microporous film, membrane, or a substrate with microreplicated surface(s). If the solid phase material includes particles, they are preferably uniform, spherical, and rigid to ensure good fluid flow characteristics.

For flow-through applications of the present invention, such materials are typically in the form of a loose, porous network to allow uniform and unimpaired entry and exit of large molecules and to provide a large surface area. Preferably, for such applications, the solid phase material has a relatively high surface area, such as, for example, more than one meter squared per gram (m²/g). For applications that do not involve the use of a flow-through device, the solid phase material may or may not be in a porous matrix. Thus, membranes can also be useful in certain methods of the present invention.

For applications that use particles or beads, they may be introduced to the sample or the sample introduced into a bed of particles/beads and removed therefrom by centrifuging, for example. Alternatively, particles/beads can be coated (e.g., pattern coated) onto an inert substrate (e.g., polycarbonate or polyethylene), optionally coated with an adhesive, by a variety of methods (e.g., spray drying). If desired, the substrate can be microreplicated for increased surface area and enhanced clean-up. It can also be pretreated with oxygen plasma, e-beam or ultraviolet radiation, heat, or a corona treatment process. This substrate can be used, for example, as a cover film, or laminated to a cover film, on a reservoir in a microfluidic device.

In one embodiment, the solid phase material includes a fibril matrix, which may or may not have particles enmeshed therein. The fibril matrix can include any of a wide variety of fibers. Typically, the fibers are insoluble in an aqueous environment. Examples include glass fibers, polyolefin fibers, particularly polypropylene and polyethylene microfibers, aramid fibers, a fluorinated polymer, particularly, polytetrafluoroethylene fibers, and natural cellulosic fibers. Mixtures of fibers can be used, which may be active or inactive toward binding of nucleic acid. Preferably, the fibril matrix forms a web that is at least about 15 microns, and no greater than about 1 millimeter, and more preferably, no greater than about 500 microns thick.

If used, the particles are typically insoluble in an aqueous environment. They can be made of one material or a combination of materials, such as in a coated particle. They can be swellable or nonswellable, although they are preferably nonswellable in water and organic liquids. Preferably, if the particle is doing the adhering, it is made of nonswelling, hydrophobic material. They can be chosen for their affinity for the nucleic acid. Examples of some water swellable particles are described in U.S. Pat. No. 4,565,663 (Errede et al.), U.S. Pat. No. 4,460,642 (Errede et al.), and U.S. Pat. No. 4,373,519 (Errede et al.). Particles that are nonswellable in water are described in U.S. Pat. No. 4,810,381 (Hagen et al.), U.S. Pat. No. 4,906,378 (Hagen et al.), U.S. Pat. No. 4,971,736 (Hagen et al.); and U.S. Pat. No. 5,279,742 (Markell et al.). Preferred particles are polyolefin particles, such as polypropylene particles (e.g., powder). Mixtures of particles can be used, which may be active or inactive toward binding of nucleic acid.

If coated particles are used, the coating is preferably an aqueous- or organic-insoluble, nonswellable material. The coating may or may not be one to which nucleic acid will adhere. Thus, the base particle that is coated can be inorganic or organic. The base particles can include inorganic oxides such as silica, alumina, titania, zirconia, etc., to which are covalently bonded organic groups. For example, covalently bonded organic groups such as aliphatic groups of varying chain length (C2, C4, C8, or C18 groups) can be used.

Examples of suitable solid phase materials that include a fibril matrix are described in U.S. Pat. No. 5,279,742 (Markell et al.), U.S. Pat. No. 4,906,378 (Hagen et al.), U.S. Pat. No. 4,153,661 (Ree et al.), U.S. Pat. No. 5,071,610 (Hagen et al.), U.S. Pat. No. 5,147,539 (Hagen et al.), 5,207,915 (Hagen et al.), and U.S. Pat. No. 5,238,621 (Hagen et al.). Such materials are commercially available from 3M Company (St. Paul, Minn.) under the trade designations SDB-RPS (Styrene-Divinyl Benzene Reverse Phase Sulfonate, 3M Part No. 2241), cation-SR membrane (3M Part No. 2251), C-8 membrane (3M Part No. 2214), and anion-SR membrane (3M Part No. 2252).

Those that include a polytetrafluoroethylene matrix (PTFE) are particularly preferred. For example, U.S. Pat. No. 4,810,381 (Hagen et al.) discloses a solid phase material that includes: a polytetrafluoroethylene fibril matrix, and nonswellable sorptive particles enmeshed in the matrix, wherein the ratio of nonswellable sorptive particles to polytetrafluoroethylene being in the range of 19:1 to 4:1 by weight, and further wherein the composite solid phase material has a net surface energy in the range of 20 to 300 milliNewtons per meter. U.S. Pat. No. RE 36,811 (Markell et al.) discloses a solid phase extraction medium that includes: a PTFE fibril matrix, and sorptive particles enmeshed in the matrix, wherein the particles include more than 30 and up to 100 weight percent of porous organic particles, and less than 70 to 0 weight percent of porous (organic-coated or uncoated) inorganic particles, the ratio of sorptive particles to PTFE being in the range of 40:1 to 1:4 by weight.

Particularly preferred solid phase materials are available under the trade designation EMPORE from the 3M Company, St. Paul, Minn. The fundamental basis of the EMPORE technology is the ability to create a particle-loaded membrane, or disk, using any sorbent particle. The particles are tightly held together within an inert matrix of polytetrafluoroethylene (90% sorbent: 10% PTFE, by weight). The PTFE fibrils do not interfere with the activity of the particles in any way. The EMPORE membrane fabrication process results in a denser, more uniform extraction medium than can be achieved in a traditional Solid Phase Extraction (SPE) column or cartridge prepared with the same size particles.

In another preferred embodiment, the solid phase (e.g., a microporous thermoplastic polymeric support) has a microporous structure characterized by a multiplicity of spaced, randomly dispersed, nonuniform shaped, equiaxed particles of thermoplastic polymer connected by fibrils. Particles are spaced from one another to provide a network of micropores therebetween. Particles are connected to each other by fibrils, which radiate from each particle to the adjacent particles. Either, or both, the particles or fibrils may be hydrophobic. Examples of preferred such materials have a high surface area, often as high as 40 meters²/gram as measured by Hg surface area techniques and pore sizes up to about 5 microns.

This type of fibrous material can be made by a preferred technique that involves the use of induced phase separation. This involves melt blending a thermoplastic polymer with an immiscible liquid at a temperature sufficient to form a homogeneous mixture, forming an article from the solution into the desired shape, cooling the shaped article so as to induce phase separation of the liquid and the polymer, and to ultimately solidify the polymer and remove a substantial portion of the liquid leaving a microporous polymer matrix. This method and the preferred materials are described in detail in U.S. Patent No. 4,726,989 (Mrozinski), U.S. Pat. No. 4,957,943 (McAllister et al.), and U.S. Pat. No. 4,539,256 (Shipman). Such materials are referred to as thermally induced phase separation membranes (TIPS membranes) and are particularly preferred.

Other suitable solid phase materials include nonwoven materials as disclosed in U.S. Pat. No. 5,328,758 (Markell et al.). This material includes a compressed or fused particulate-containing nonwoven web (preferably blown microfibrous) that includes high sorptive-efficiency chromatographic grade particles.

Other suitable solid phase materials include those known as HIPE Foams, which are described, for example, in U.S. Pat. Publication No. 2003/0011092 (Tan et al.). “HIPE” or “high internal phase emulsion” means an emulsion that includes a continuous reactive phase, typically an oil phase, and a discontinuous or co-continuous phase immiscible with the oil phase, typically a water phase, wherein the immiscible phase includes at least 74 volume percent of the emulsion. Many polymeric foams made from HIPE's are typically relatively open-celled. This means that most or all of the cells are in unobstructed communication with adjoining cells. The cells in such substantially open-celled foam structures have intercellular windows that are typically large enough to permit fluid transfer from one cell to another within the foam structure.

The solid phase material can include capture sites for inhibitors. Herein, “capture sites” refer to groups that are either covalently attached (e.g., functional groups) or molecules that are noncovalently (e.g., hydrophobically) attached to the solid phase material.

Preferably, the solid phase material includes functional groups that capture the inhibitors. For example, the solid phase material may include chelating groups. In this context, “chelating groups” are those that are polydentate and capable of forming a chelation complex with a metal atom or ion (although the inhibitors may or may not be retained on the solid phase material through a chelation mechanism). The incorporation of chelating groups can be accomplished through a variety of techniques. For example, a nonwoven material can hold beads functionalized with chelating groups. Alternatively, the fibers of the nonwoven material can be directly functionalized with chelating groups.

Examples of chelating groups include, for example, —(CH₂—C(O)OH)₂, tris(2-aminoethyl)amine groups, iminodiacetic acid groups, nitrilotriacetic acid groups. The chelating groups can be incorporated into a solid phase material through a variety of techniques. They can be incorporated in by chemically synthesizing the material. Alternatively, a polymer containing the desired chelating groups can be coated (e.g., pattern coated) on an inert substrate (e.g., polycarbonate or polyethylene). If desired, the substrate can be microreplicated for increased surface area and enhanced clean-up. It can also be pretreated with oxygen plasma, e-beam or ultraviolet radiation, heat, or a corona treatment process. This substrate can be used, for example, as a cover film, or laminated to a cover film, on a reservoir in a microfluidic device.

Chelating solid phase materials are commercially available and could be used as the solid phase material in the present invention. For example, for certain embodiments of the present invention, EMPORE membranes that include chelating groups such as iminodiacetic acid (in the form of the sodium salt) are preferred. Examples of such membranes are disclosed in U.S. Pat. No. 5,147,539 (Hagen et al.) and commercially available as EMPORE Extraction Disks (47 mm, No. 2271 or 90 mm, No. 2371) from the 3M Company. For certain embodiments of the present invention, ammonium-derivatized EMPORE membranes that include chelating groups are preferred. To put the disk in the ammonium form, it can be washed with 50 mL of 0.1M ammonium acetate buffer at pH 5.3 followed with several reagent water washes.

Examples of other chelating materials include, but are not limited to, crosslinked polystyrene beads available under the trade designation CHELEX from Bio-Rad Laboratories, Inc. (Hercules, Calif.), crosslinked agarose beads with tris(2-aminoethyl)amine, iminodiacetic acid, nitrilotriacetic acid,polyamines and polyimines as well as the chelating ion exchange resins commercially available under the trade designation DUOLITE C-467 and DUOLITE GT73 from Rohm and Haas (Philadelphia, Pa.), AMBERLITE IRC-748, DIAION CR11, DUOLITE C647.

CHELEX 100 chelating resin, a styrene divinyl benzene copolymer containing iminodiacetate groups (—N—(CH₂—C(O)OH)₂), has a high affinity for polyvalent metal ions and can be used in certain methods of the present invention, although it is less desirable in methods carried out in microfluidic devices.

Typically, a desired concentration density of chelating groups on the solid phase material is about 0.02 nanomole per millimeter squared, although it is believed that a wider range of concentration densities is possible.

Other types of capture materials include anion exchange materials, cation exchange materials, activated carbon, reverse phase, normal phase, styrene-divinyl benzene, alumina, silica, zirconia, metal colloids. Examples of suitable anion exchange materials include strong anion exchangers such as quaternary ammonium, dimethylethanolamine, quaternary alkylamine, trimethylbenzyl ammonium, and dimethylethanolbenzyl ammonium usually in the chloride form, and weak anion exchangers such as polyamine. Examples of suitable cation exchange materials include strong cation exchangers such as sulfonic acid typically in the sodium form, and weak cation exchangers such as carboxylic acid typically in the hydrogen form. Examples of suitable carbon-based materials include EMPORE carbon materials, carbon beads, Examples of suitable reverse phase C8 and C18 materials include silica beads that are end-capped with octadecyl groups or octyl groups and EMPORE materials that have C8 and C18 silica beads (EMPORE materials are available from 3M Co., St. Paul, Minn.). Examples of normal phase materials include hydroxy groups and dihydroxy groups.

Commercially available materials can also be modified or directly used in methods of the present invention, particularly in microfluidic devices. For example, solid phase materials available under the trade designation LYSE AND GO (Pierce, Rockford, Ill.), RELEASE-IT (CPG, NJ), GENE FIZZ (Eurobio, France), GENE RELEASER (Bioventures Inc., Murfreesboro, Tenn.), and BUGS N BEADS (GenPoint, Oslo, Norway), as well as Zymo's beads (Zymo Research, Orange, Calif.) and Dynal's beads (Dynal, Oslo, Norway) can be incorporated into the methods of the present invention, particularly into a microfluidic device as the solid phase capture material.

In certain embodiments of such methods, the solid phase material includes a coating reagent. The coating reagent is preferably selected from the group consisting of a surfactant, a strong base, a polyelectrolyte, a selectively permeable polymeric barrier, and combinations thereof. In certain embodiments of such methods, the solid phase material includes a polytetrafluoroethylene fibril matrix, sorptive particles enmeshed in the matrix, and a coating reagent coated on the solid phase material, wherein the coating reagent is selected from the group consisting of a surfactant, a strong base, a polyelectrolyte, a selectively permeable polymeric barrier, and combinations thereof.

Examples of suitable surfactants are listed below.

Examples of suitable strong bases include NaOH, KOH, LiOH, NH₄OH, as well as primary, secondary, or tertiary amines.

Examples of suitable polyelectrolytes include, polystryene sulfonic acid (e.g., poly(sodium 4-styrenesulfonate) or PSSA), polyvinyl phosphonic acid, polyvinyl boric acid, polyvinyl sulfonic acid, polyvinyl sulfuric acid, polystyrene phosphonic acid, polyacrylic acid, polymethacrylic acid, lignosulfonate, carrageenan, heparin, chondritin sulfate, and salts or other derivatives thereof.

Examples of suitable selectively permeable polymeric barriers include polymers such as acrylates, acryl amides, azlactones, polyvinyl alcohol, polyethylene imine, polysaccharides. Such polymers can be in a variety of forms. They can be water-soluble, water-swellable, water-insoluble, hydrogels, etc. For example, a polymeric barrier can be prepared such that it acts as a filter for larger particles such as white blood cells, nuclei, viruses, bacteria, as well as nucleic acids such as human genomic DNA and proteins. These surfaces could be tailored by one of skill in the art to separate on the basis of size and/or charge by appropriate selection of functional groups, by cross-linking, and the like. Such materials would be readily available or prepared by one of skill in the art.

Preferably, the solid phase material is coated with a surfactant without washing any surfactant excess away, although the other coating reagents can be rinsed away if desired. Typically, the coating can be carried out using a variety of methods such as dipping, rolling, spraying, etc. The coating reagent-loaded solid phase material is then typically dried, for example, in air, prior to use.

Particularly desirable are solid phase materials that are coated with a surfactant, preferably a nonionic surfactant. This can be accomplished according to the procedure set forth in the Examples Section. Although not intending to be limited by theory, the addition of the surfactant is believed to increase the wettability of the solid phase material, which allows the inhibitors to soak into the solid phase material and bind thereto.

The coating reagent for the solid phase materials are preferably aqueous-based solutions, although organic solvents (alcohols, etc.) can be used, if desired. The coating reagent loading should be sufficiently high such that the sample is able to wet out the solid phase material. It should not be so high, however, that there is significant elution of the coating reagent itself. Preferably, if the coating reagent is eluted with the nucleic acid, there is no more than about 2 wt-% coating reagent in the eluted sample. Typically, the coating solution concentrations can be as low as 0.1 wt-% coating reagent in the solution and as high as 10 wt-% coating reagent in the solution.

### Surfactants

Nonionic Surfactants. A wide variety of suitable nonionic surfactants are known that can be used as a lysing reagent (discussed above), an eluting reagent (discussed below), and/or as a coating on the solid phase material. They include, for example, polyoxyethylene surfactants, carboxylic ester surfactants, carboxylic amide surfactants, etc. Commercially available nonionic surfactants include, n-dodecanoylsucrose, n-dodecyl-β-D-glucopyranoside, n-octyl-β-D-maltopyranoside, n-octyl-β-D-thioglucopyranoside, n-decanoylsucrose, n-decyl-β-D-maltopyranoside, n-decyl-β-D-thiomaltoside, n-heptyl-β-D-glucopyranoside, n-heptyl-β-D-thioglucopyranoside, n-hexyl-β-D-glucopyranoside, n-nonyl-β-D-glucopyranoside, n-octanoylsucrose, n-octyl-β-D-glucopyranoside, cyclohexyl-n-hexyl-β-D-maltoside, cyclohexyl-n-methyl-β-D-maltoside, digitonin, and those available under the trade designations PLURONIC, TRITON, TWEEN, as well as numerous others commercially available and listed in the Kirk Othmer Technical Encyclopedia. Examples are listed in Table 1 below. Preferred surfactants are the polyoxyethylene surfactants. More preferred surfactants include octyl phenoxy polyethoxyethanol.

TABLE 1

| SURFACTANT | | |
|---|---|---|
| TRADE NAME | NONIONIC SURFACTANT | SUPPLIER |
| PLURONIC F127 | Modified oxyethylated alcohol and/or | Sigma |
| | oxypropylated straight chain alcohols | St. Louis, MO |
| TWEEN 20 | Polyoxyethylene (20) sorbitan | Sigma |
| | monolaurate | St. Louis, MO |
| TRITON X-100 | t-Octyl phenoxy polyethoxyethanol | Sigma |
| | | St. Louis, MO |
| BRIJ 97 | Polyoxyethylene (10) oleyl ether | Sigma |
| | | St. Louis, MO |
| IGEPAL CA-630 | Octyl phenoxy poly (ethyleneoxy) | Sigma |
| | ethanol | St. Louis, MO |
| TOMADOL 1-7 | Ethoxylated alcohol | Tomah |
| | | Products |
| | | Milton, WI |
| Vitamin E TPGS | d-Alpha tocopheryl polyethylene | Eastman |
| | glycol 1000 | Kingsport, TN |

Also suitable are fluorinated nonionic surfactants of the type disclosed in U.S. Pat. Publication Nos. 2003/0139550 (Savu et al.) and 2003/0139549 (Savu et al.). Other nonionic fluorinated surfactants include those available under the trade designation ZONYL from DuPont (Wilmington, Del.).

Zwitterionic Surfactants. A wide variety of suitable zwitterionic surfactants are known that can be used as a coating on the solid phase material, as a lysing reagent, and/or as an eluting reagent. They include, for example, alkylamido betaines and amine oxides thereof, alkyl betaines and amine oxides thereof, sulfo betaines, hydroxy sulfo betaines, amphoglycinates, amphopropionates, balanced amphopolycarboxyglycinates, and alkyl polyaminoglycinates. Proteins have the ability of being charged or uncharged depending on the pH; thus, at the right pH, a protein, preferably with a pI of about 8 to 9, such as modified Bovine Serum Albumin or chymotrypsinogen, could function as a zwitterionic surfactant. A specific example of a zwitterionic surfactant is cholamido propyl dimethyl ammonium propanesulfonate available under the trade designation CHAPS from Sigma. More preferred surfactants include N-dodecyl-N,N dimethyl-3-ammonia-1-propane sulfonate.

Cationic Surfactants. A wide variety of suitable cationic surfactants are known that can be used as a lysing reagent, an eluting reagent, and/or as a coating on the solid phase material. They include, for example, quaternary ammonium salts, polyoxyethylene alkylamines, and alkylamine oxides. Typically, suitable quaternary ammonium salts include at least one higher molecular weight group and two or three lower molecular weight groups are linked to a common nitrogen atom to produce a cation, and wherein the electrically-balancing anion is selected from the group consisting of a halide (bromide, chloride, etc.), acetate, nitrite, and lower alkosulfate (methosulfate, etc.). The higher molecular weight substituent(s) on the nitrogen is/are often (a) higher alkyl group(s), containing about 10 to about 20 carbon atoms, and the lower molecular weight substituents may be lower alkyl of about 1 to about 4 carbon atoms, such as methyl or ethyl, which may be substituted, as with hydroxy, in some instances. One or more of the substituents may include an aryl moiety or may be replaced by an aryl, such as benzyl or phenyl. Among the possible lower molecular weight substituents are also lower alkyls of about 1 to about 4 carbon atoms, such as methyl and ethyl, substituted by lower polyalkoxy moieties such as polyoxyethylene moieties, bearing a hydroxyl end group, and falling within the general formula:
R(CH₂CH₂O)₍ₙ₋₁₎CH₂CH₂OH
where R is a (C1-C4)divalent alkyl group bonded to the nitrogen, and n represents an integer of about 1 to about 15. Alternatively, one or two of such lower polyalkoxy moieties having terminal hydroxyls may be directly bonded to the quaternary nitrogen instead of being bonded to it through the previously mentioned lower alkyl. Examples of useful quaternary ammonium halide surfactants for use in the present invention include but are not limited to methyl-bis(2-hydroxyethyl)coco-ammonium chloride or oleyl-ammonium chloride, (ETHOQUAD C/12 and O/12, respectively) and methyl polyoxyethylene (15) octadecyl ammonium chloride (ETHOQUAD 18/25) from Akzo Chemical Inc.

Anionic Surfactants. A wide variety of suitable anionic surfactants are known that can be used as a lysing reagent, an eluting reagent, and/or as a coating on the solid phase material. Surfactants of the anionic type that are useful include sulfonates and sulfates, such as alkyl sulfates, alkylether sulfates, alkyl sulfonates, alkylether sulfonates, alkylbenzene sufonates, alkylbenzene ether sulfates, alkylsulfoacetates, secondary alkane sulfonates, secondary alkylsulfates and the like. Many of these can include polyalkoxylate groups (e.g., ethylene oxide groups and/or propylene oxide groups, which can be in a random, sequential, or block arrangement) and/or cationic counterions such as Na, K, Li, ammonium, a protonated tertiary amine such as triethanolamine or a quaternary ammonium group. Examples include: alkyl ether sulfonates such as lauryl ether sulfates available under the trade designation POLYSTEP B12 and B22 from Stepan Company, Northfield, Ill., and sodium methyl taurate available under the trade designation NIKKOL CMT30 from Nikko Chemicals Co., Tokyo, Japan); secondary alkane sulfonates available under the trade designation HOSTAPUR SAS, which is a sodium (C14-C17)secondary alkane sulfonates (alpha-olefin sulfonates), from Clariant Corp., Charlotte, N.C.; methyl-2-sulfoalkyl esters such as sodium methyl-2-sulfo(C12-C16)ester and disodium 2-sulfo(C12-C16)fatty acid available from Stepan Company under the trade designation ALPHASTE PC-48; alkylsulfoacetates and alkylsulfosuccinates available as sodium laurylsulfoacetate (trade designation LANTHANOL LAL) and disodiumlaurethsulfosuccinate (trade designation STEPANMILD SL3), both from Stepan Co.; and alkylsulfates such as ammoniumlauryl sulfate commercially available under the trade designation STEPANOL AM from Stepan Co.

Another class of useful anionic surfactants include phosphates such as alkyl phosphates, alkylether phosphates, aralkylphosphates, and aralkylether phosphates. Many of these can include polyalkoxylate groups (e.g., ethylene oxide groups and/or propylene oxide groups, which can be in a random, sequential, or block arrangement). Examples include a mixture of mono-, di- and tri-(alkyltetraglycolether)-o-phosphoric acid esters generally referred to as trilaureth-4-phosphate commercially available under the trade designation HOSTAPHAT 340KL from Clariant Corp., and PPG-5 ceteth 10 phosphate available under the trade designation CRODAPHOS SG from Croda Inc., Parsipanny, N.J., as well as alkyl and alkylamidoalkyldialkylamine oxides. Examples of amine oxide surfactants include those commercially available under the trade designations AMMONYX LO, LMDO, and CO, which are lauryldimethylamine oxide, laurylamidopropyldimethylamine oxide, and cetyl amine oxide, all from Stepan Co.

### Elution Techniques

For embodiments that use a solid phase material for retaining inhibitors, the more concentrated region of the sample that includes nucleic acid-containing material (e.g., nuclei) and/or released nucleic acid can be eluted using a variety of eluting reagents. Such eluting reagents can include water (preferably RNAse-free sterile water), a buffer, a surfactant, which can be cationic, anionic, nonionic, or zwitterionic, or a strong base.

Preferably the eluting reagent is basic (i.e., greater than 7). For certain embodiments, the pH of the eluting reagent is at least 8. For certain embodiments, the pH of the eluting reagent is up to 10. For certain embodiments, the pH of the eluting reagent is up to 13. If the eluted nucleic acid is used directly in an amplification process such as PCR, the eluting reagent should be formulated so that the concentration of the ingredients will not inhibit the enzymes (e.g., Taq Polymerase) or otherwise prevent the amplification reaction.

Examples of suitable surfactants include those listed above, particularly, those known as SDS, TRITON X-100, TWEEN, fluorinated surfactants, and PLURONICS. The surfactants are typically provided in aqueous-based solutions, although organic solvents (alcohols, etc.) can be used, if desired. The concentration of a surfactant in an eluting reagent is preferably at least 0.1 weight/volume percent (w/v-%), based on the total weight of the eluting reagent. The concentration of a surfactant in an eluting reagent is preferably no greater than 1 w/v-%, based on the total weight of the eluting reagent. A stabilizer, such as polyethylene glycol, can optionally be used with a surfactant.

Examples of suitable elution buffers include TRIS-HCl, N-[2-hydroxyethyl]piperazine-N′-[2-ethanesulfonic acid] (HEPES), 3-[N-morpholino]propanesulfonic acid (MOPS), piperazine-N,N′-bis[2-ethanesulfonic acid] (PIPES), 2-[N-morpholino]ethansulfonic acid (MES), TRIS-EDTA (TE) buffer, sodium citrate, ammonium acetate, carbonate salts, and bicarbonates etc.

The concentration of an elution buffer in an eluting reagent is preferably at least 10 millimolar (mM). The concentration of a surfactant in an eluting reagent is preferably no greater than 2 weight percent (wt-%).

Typically, elution of the nucleic acid-containing material and/or released nucleic acid is preferably accomplished using an alkaline solution. Although not intending to be bound by theory, it is believed that an alkaline solution allows for improved binding of inhibitors, as compared to elution with water. The alkaline solution also facilitates lysis of nucleic acid-containing material. Preferably, the alkaline solution has a pH of 8 to 13, and more preferably 13. Examples of sources of high pH include aqueous solutions of NaOH, KOH, LiOH, quaternary nitrogen base hydroxide, tertiary, secondary or primary amines, etc. If an alkaline solution is used for elution, it is typically neutralized in a subsequent step, for example, with TRIS buffer, to form a PCR-ready sample.

The use of an alkaline solution can selectively destroy RNA, to allow for the analysis of DNA. Otherwise, RNAse can be added to the formulation to inactivate RNA, followed by heat inactivation of the RNAse. Similarly, DNAse can be added to selectively destroy DNA and allow for the analysis of RNA; however, other lysis buffers (e.g., TE) that do not destroy RNA would be used in such methods. The addition of RNAse inhibitor such as RNAsin can also be used in a formulation for an RNA preparation that is subjected to real-time PCR.

Elution is typically carried out at room temperature, although higher temperatures may produce higher yields. For example, the temperature of the eluting reagent can be up to 95° C. if desired. Elution is typically carried out within 10 minutes, although 1-3 minute elution times are preferred.

### Devices and Kits

Examples of devices for using the methods of the present invention include standard laboratory filter holders furnished by companies such as Millipore, Inc. (Bedford, Mass.), Bio-Rad, Inc. (Hercules, Calif.), Osmonics, Inc. (Westborough, Mass.), and Whatman, Inc. (Clifton, N.J.). The method of the invention can be conducted in filtration devices which facilitate the movement of solutions through solid phase materials (referred to as flow-through devices) by means including centrifugation, suction, pressure. Other devices include microtiter plates and microfluidic devices.

The present invention also provides a kit, which can include a solid phase material either with or without a holder (for example, a filter holder such as a syringe filter holder or a spin filter holder, or a column with retaining frits at each end for retaining particulate material), a lysing reagent (particularly a surfactant such as a nonionic surfactant, either neat or in a solution), and instructions for binding inhibitors and eluting the nucleic acid. Preferably, the present invention provides kits that include a flow-through receptacle (more preferably, a microfluidic device) having a solid phase (preferably, polymeric) material therein, and preferably a nonionic surfactant.

Other components that could be included within kits of the present invention include conventional reagents such as wash solutions, coupling buffers, quenching buffers, blocking buffers, elution buffers, and the like. Other components that could be included within kits of the present invention include conventional equipment such as spin columns, cartridges, 96-well filter plates, syringe filters, collection units, syringes, and the like.

The kits typically include packaging material, which refers to one or more physical structures used to house the contents of the kit. The packaging material can be constructed by well-known methods, preferably to provide a sterile, contaminant-free environment. The packaging material may have a label that indicates the contents of the kit. In addition, the kit contains instructions indicating how the materials within the kit are employed. As used herein, the term “package” refers to a solid matrix or material such as glass, plastic, paper, foil, and the like.

“Instructions” typically include a tangible expression describing the various methods of the present invention, including lysing conditions (e.g., lysing reagent type and concentration), the relative amounts of reagent and sample to be admixed, maintenance time periods for reagent/sample admixtures, temperature, buffer conditions, and the like.

Although the methods can be used in a variety of devices, a variety of illustrative embodiments of preferred devices are described in U.S. Patent Publication Nos. 2002/0047003 (published Apr. 25, 2003, Bedingham et al.).

Preferred devices useful in methods of the present invention include microfluidic devices. These typically employ a body structure that has an integrated microfluidic channel network disposed therein. In preferred aspects, the body structure of the microfluidic devices include an aggregation of two or more separate layers which, when appropriately mated or joined together, form the microfluidic device of the invention, e.g., containing the channels and/or chambers described herein. Typically, useful microfluidic devices include a top portion, a bottom portion, and an interior portion, wherein the interior portion substantially defines the channels and chambers of the device. Typically, the chambers include valves (e.g., valve septums) and are referred to as valved chambers.

A particularly preferred device for certain embodiments herein is referred to as a variable valve device and is disclosed in Applicants' Assignee's copending U.S. patent application Ser. No. 10/734,717, filed on Dec. 12, 2003, entitled Variable Valve Apparatus and Method. In this variable valve device, the valve structures allow for removal of selected portions of the sample material located within the process chamber (i.e., the variable valved process chamber). Removal of the selected portions is achieved by forming an opening in a valve septum at a desired location.

The valve septums are preferably large enough to allow for adjustment of the location of the opening based on the characteristics of the sample material in the process chamber. If the sample processing device is rotated after the opening is formed, the selected portion of the material located closer to the axis of rotation exits the process chamber through the opening. The remainder of the sample material cannot exit through the opening because it is located farther from the axis of rotation than the opening.

The openings in the valve septum may be formed at locations based on one or more characteristics of the sample material detected within the process chamber. It may be preferred that the process chambers include detection windows that transmit light into and/or out of the process chamber. Detected characteristics of the sample material may include, e.g., the free surface of the sample material (indicative of the volume of sample material in the process chamber). Forming an opening in the valve septum at a selected distance radially outward of the free surface can provide the ability to remove a selected volume of the sample material from the process chamber.

For sample materials that can be separated into various components, e.g., whole blood, rotation of the sample processing device may result in separation of the plasma and red blood cell components, thus allowing for selective removal of the components to, e.g., different process chambers.

In some embodiments, it may be possible to remove selected aliquots of the sample material by forming openings at selected locations in one or more valve septums. The selected aliquot volume can be determined based on the radial distance between the openings (measured relative to the axis of rotation) and the cross-sectional area of the process chamber between the opening.

The openings in the valve septums are preferably formed in the absence of physical contact, e.g., through laser ablation, focused optical heating, etc. As a result, the openings can preferably be formed without piercing the outermost layers of the sample processing device, thus limiting the possibility of leakage of the sample material from the sample processing device.

In one aspect, the present invention uses a valved process chamber in a sample processing device (e.g., a microfluidic device), the valved process (e.g., heating, mixing, lysing, combining fluids) chamber including a process chamber having a process chamber volume located between opposing first and second major sides of the sample processing device, wherein the process chamber occupies a process chamber area in the sample processing device, and wherein the process chamber area has a length and a width transverse to the length, and further wherein the length is greater than the width. The variable valved process chamber also includes a valve chamber located within the process chamber area, the valve chamber located between the process chamber volume and the second major side of the sample processing device, wherein the valve chamber is isolated from the process chamber by a valve septum separating the valve chamber and the process chamber, and wherein a portion of the process chamber volume lies between the valve septum and a first major side of the sample processing device. A detection window is located within the process chamber area, wherein the detection window is transmissive to selected electromagnetic energy directed into and/or out of the process chamber volume.

In another aspect, the present invention provides a method that allows for the selective removal of a portion of a sample from a variable valved process chamber. The method includes providing a sample processing device (e.g., a microfluidic device) as described above, providing sample material in the process chamber; detecting a characteristic of the sample material in the process chamber through the detection window; and forming an opening in the valve septum at a selected location along the length of the process chamber, wherein the selected location is correlated to the detected characteristic of the sample material. The method also includes moving only a portion of the sample material from the process chamber into the valve chamber through the opening formed in the valve septum

### Illustrative Method 1

In one embodiment, the present invention provides a method of isolating nucleic acid from a sample. In this illustrative method, the inhibitors are contained within cells, but it is understood that this may not always be the case, and methods of the present invention can be used with such samples.

The method includes: providing a sample including nucleic acid-containing material and cells containing inhibitors (such nucleic acid-containing material and cells containing inhibitors may be the same or different); contacting the sample with a first lysing reagent under conditions effective to break cell membranes and release inhibitors and form a lysed sample including nucleic acid-containing material and inhibitors; forming a concentrated region of the lysed sample; wherein the concentrated region of the lysed sample includes nucleic acid-containing material and inhibitors; substantially separating the concentrated region from a less concentrated region of the lysed sample; contacting the separated concentrated region of the lysed sample with a solid phase material to preferentially adhere at least a portion of the inhibitors to the solid phase material, wherein the solid phase material includes a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof; optionally further lysing the nucleic acid-containing material to release nucleic acid before, simultaneous with, or after contacting the separated concentrated region of the sample with the solid phase material; and separating at least a portion of the nucleic acid-containing material and/or nucleic acid from the solid phase material having at least a portion of the inhibitors adhered thereto.

The nucleic acid-containing material and cells containing inhibitors may be the same or different, although they are typically different. That is, the nucleic acid containing material and the inhibitor-containing cells could potentially be the same. For example, if the sample is a buffy coat, the nucleic acid containing material can be a white blood cell, which includes both nuclei and inhibitors. If a lysing reagent (e.g., a nonionic surfactant) is used that will lyse the cell membranes of the white blood cells but not the nuclei included therein, then the inhibitors are released as are in-tact nuclei, which is also considered to be nucleic acid-containing material as defined herein.

In another embodiment, the present invention provides a method of isolating nucleic acid from a sample, the method includes: providing a sample including cells containing inhibitors and cells containing nuclei (such cells containing inhibitors and cells containing nuclei may be the same or different); contacting the biological sample with a nonionic surfactant under conditions effective to break cell membranes and release nuclei and inhibitors and form a lysed sample; forming a concentrated region of the lysed sample; wherein the concentrated region of the sample includes nuclei and inhibitors; substantially separating the concentrated region of the lysed sample from the less concentrated region of the sample; contacting the separated concentrated region of the lysed sample with a solid phase material to preferentially adhere at least a portion of the inhibitors to the solid phase material, wherein the solid phase material includes a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof; optionally further lysing the nuclei to release nucleic acid before, simultaneous with, or after contacting the separated concentrated region of the sample with the solid phase material; and separating at least a portion of the nucleic acid-containing and/or nucleic acid from the solid phase material having at least a portion of the inhibitors adhered thereto.

The cells containing inhibitors and cells containing nuclei may be the same or different, although they are typically different cells. That is, the nuclei-containing cells and the inhibitor-containing cells could potentially be the same. For example, the inhibitors could include nuclear proteins, as well as cellular proteins.

In another embodiment, the present invention provides a method of isolating nucleic acid from a sample, the method includes: providing a microfluidic device including a loading chamber, a valved process chamber, and a separation chamber including a solid phase material; providing a sample including nucleic acid-containing material and cells containing inhibitors (such nucleic acid-containing material and cells containing inhibitors may be the same or different); placing the sample in the loading chamber; contacting the sample with a first lysing reagent under conditions effective to break cell membranes and release inhibitors and form a lysed sample including nucleic acid-containing material and inhibitors; transferring the lysed sample to a valved process chamber; forming a concentrated region of the lysed sample in the valved process chamber, wherein the concentrated region of the lysed sample includes nucleic acid-containing material and inhibitors; substantially separating the concentrated region from a less concentrated region of the lysed sample; transferring the separated concentrated region of the lysed sample to the separation chamber for contact with the solid phase material to preferentially adhere at least a portion of the inhibitors to the solid phase material; wherein the solid phase material includes capture sites (preferably chelating functional groups), a coating reagent coated on the solid phase material, or both; wherein the coating reagent is selected from the group consisting of a surfactant, a strong base, a polyelectrolyte, a selectively permeable polymeric barrier, and combinations thereof; optionally further lysing the nucleic acid-containing material to release nucleic acid before, simultaneous with, or after contacting the separated concentrated region of the sample with the solid phase material; and separating at least a portion of the nucleic acid-containing material and/or nucleic acid from the solid phase material having at least a portion of the inhibitors adhered thereto. As discussed above, the nucleic acid-containing material and cells containing inhibitors may be the same or different, although they are typically different.

In another embodiment, the present invention provides a method of isolating nucleic acid from a sample, the method includes: providing a microfluidic device including a loading chamber, a valved process chamber, and a separation chamber including a solid phase material; providing a sample including cells containing inhibitors and cells containing nuclei (such cells containing inhibitors and cells containing nuclei may be the same or different); placing the sample in the loading chamber; contacting the sample with a nonionic surfactant under conditions effective to break cell membranes and release nuclei and inhibitors to form a lysed sample; transferring the lysed sample to a valved process chamber; forming a concentrated region of the lysed sample in the valved process chamber, wherein the concentrated region of the lysed sample includes nuclei and inhibitors; substantially separating the concentrated region from a less concentrated region of the lysed sample; transferring the separated concentrated region of the lysed sample to the separation chamber for contact with the solid phase material to preferentially adhere at least a portion of the inhibitors to the solid phase material; wherein the solid phase material includes capture sites (preferably chelating functional groups), a coating reagent coated on the solid phase material, or both; wherein the coating reagent is selected from the group consisting of a surfactant, a strong base, a polyelectrolyte, a selectively permeable polymeric barrier, and combinations thereof; optionally further lysing the nuclei to release nucleic acid before, simultaneous with, or after contacting the separated concentrated region of the sample with the solid phase material; and separating at least a portion of the nucleic acid-containing and/or nucleic acid from the solid phase material having at least a portion of the inhibitors adhered thereto. As discussed above, the cells containing inhibitors and cells containing nuclei may be the same or different, although they are typically different cells.

In certain embodiments of such methods, the nucleic acid-containing material includes nuclei and the method includes separating at least a portion of the nuclei from the solid phase material. That is, in-tact nuclei contact the solid phase material.

In certain embodiments of such methods, separating at least a portion of the nucleic acid-containing material (e.g., nuclei) and/or nucleic acid from the solid phase material includes transferring the nucleic acid-containing material (e.g., nuclei) and/or nucleic acid to an amplification reaction chamber (e.g., PCR sample chamber).

In certain embodiments of such methods, the coating reagent on the solid phase material includes a surfactant. Preferably, the surfactant is a nonionic or a zwitterionic surfactant, and more preferably, a nonionic surfactant.

In certain embodiments of such methods, separating at least a portion of the nucleic acid-containing material (e.g., nuclei) and/or nucleic acid includes eluting at least a portion of the nucleic acid-containing material (e.g., nuclei) and/or nucleic acid from the solid phase material with an eluting reagent.

In certain embodiments of such methods of the invention, if desired the methods can include further lysing the nucleic acid-containing material (e.g., nuclei) using a second lysing reagent.

Alternatively, in certain embodiments of such methods, the eluting reagent is also a lysing reagent. For elution and lysing simultaneously, a strong base, such as NaOH is preferred. For elution without lysing, water or TE buffer is preferred. For elution of a prelysed sample, any elution reagent (i.e., eluting reagent) can be used.

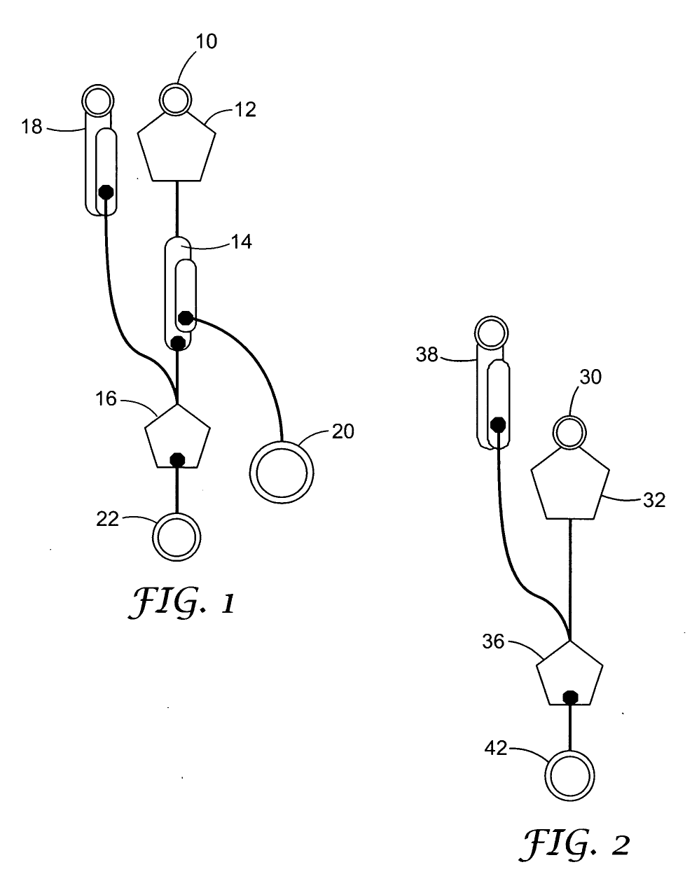

Referring to FIG. 1, a preferred embodiment of the microfluidic device suitable for use with these embodiments includes a loading chamber 10, an optional mixing chamber 12, a valved process chamber 14, a separation chamber 16 that includes a solid phase material, an eluting reagent chamber 18, a waste chamber 20 and an optional amplification reaction chamber 22. A microfluidic device that includes such a valved process chamber is disclosed, for example, in Applicants' Assignee's copending U.S. patent application Ser. No. 10/734,717, filed on Dec. 12, 2003, entitled Variable Valve Apparatus and Method. These chambers are in fluid communication with each other such that a sample can be loaded into the loading chamber 10, which can then be transferred to the mixing chamber 12, or if it is not present, directly to the valved process chamber 14. If the sample is premixed with a lysing reagent (i.e., pre-lysed), for example, then the mixing chamber 12 may not be needed. Alternatively, the mixing chamber 12 could include a lysing reagent, for example, in a pre-deposited (and typically, a dried-down) form. The sample can be concentrated in the valved process chamber 14 by a variety of means, typically by centriftigation. The valve of the valved process chamber 14 is typically positioned such that a concentrated region of a sample (that includes target nucleic acid-containing material and/or nucleic acid and inhibitors) can be substantially separated from the less concentrated region of the sample (that can also include inhibitors and a lesser amount of the target nucleic acid-containing material and/or nucleic acid). The less concentrated region of the sample is typically transferred to the waste chamber 20. The more concentrated region of the sample is transferred to the separation chamber 16 for contact with the solid phase material to preferentially adhere at least a portion of the inhibitors to the solid phase material. The eluting reagent in the eluting reagent chamber 18 is then transferred to the separation chamber 16 to remove at least a portion of the target nucleic acid-containing material and/or nucleic acid. In some cases, it may be useful to archive the nuclei in the consumable for later use. In certain embodiments, this material can be directly transferred to an amplification reaction chamber 22 for carrying out a PCR process, for example. The amplification reaction chamber 22 can optionally include pre-deposited (and typically, dried-down) reactants for the amplification reaction (e.g., PCR).

### Illustrative Method 2

In another embodiment, the present invention provides a method of isolating nucleic acid from a sample. In this illustrative method, the inhibitors are contained within cells, but it is understood that this may not always be the case, and methods of the present invention can be used with such samples.

The method includes: providing a sample including nucleic acid-containing material and cells containing inhibitors (which may be the same or different); contacting the sample with a first lysing reagent under conditions effective to break cell membranes and release inhibitors and form a lysed sample including nucleic acid-containing material and inhibitors; contacting the lysed sample with a solid phase material to preferentially adhere at least a portion of the inhibitors to the solid phase material, wherein the solid phase material includes a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof; optionally further lysing the nucleic acid-containing material to release nucleic acid before, simultaneous with, or after contacting the lysed sample with the solid phase material; and separating at least a portion of the nucleic acid-containing material and/or nucleic acid from the solid phase material having at least a portion of the inhibitors adhered thereto by eluting with an eluting reagent, with the proviso that the eluting reagent is not a nonionic surfactant.

In another embodiment, the present invention provides a method of isolating nucleic acid from a sample, the method includes: providing a sample including nucleic acid-containing material and cells containing inhibitors (which may be the same or different); contacting the sample with a first lysing reagent under conditions effective to break cell membranes and release inhibitors and form a lysed sample including nucleic acid-containing material and inhibitors; contacting the lysed sample with a solid phase material to preferentially adhere at least a portion of the inhibitors to the solid phase material, wherein the solid phase material includes a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof; separating at least a portion of the nucleic acid-containing material from the solid phase material having at least a portion of the inhibitors adhered thereto; and after separating the nucleic acid-containing material, further lysing the nucleic acid-containing material to release nucleic acid.

In another embodiment, the present invention provides a method of isolating nucleic acid from a sample, the method includes: providing a microfluidic device including a loading chamber and a separation chamber including a solid phase material; providing a sample including nucleic acid-containing material and cells containing inhibitors (which may be the same or different); placing the sample in the loading chamber; contacting the sample with a first lysing reagent under conditions effective to break cell membranes and release inhibitors and form a lysed sample including nucleic acid-containing material and inhibitors; transferring the lysed sample to the separation chamber to contact the solid phase material and to preferentially adhere at least a portion of the inhibitors to the solid phase material; optionally further lysing the nucleic acid-containing material to release nucleic acid before, simultaneous with, or after contacting the sample with the solid phase material; wherein the solid phase material includes capture sites (e.g., chelating functional groups), a coating reagent coated on the solid phase material, or both; wherein the coating reagent is selected from the group consisting of a surfactant, a strong base, a polyelectrolyte, a selectively permeable polymeric barrier, and combinations thereof, and separating at least a portion of the nucleic acid-containing material and/or nucleic acid from the solid phase material having at least a portion of the inhibitors adhered thereto by eluting with an eluting reagent, with the proviso that the eluting reagent is not a nonionic surfactant.

In another embodiment, the present invention provides a method of isolating nucleic acid from a sample, the method includes: providing a microfluidic device including a loading chamber and a separation chamber including a solid phase material; providing a sample including nucleic acid-containing material and cells containing inhibitors (which may be the same or different); placing the sample in the loading chamber; contacting the sample with a first lysing reagent under conditions effective to break cell membranes and release inhibitors and form a lysed sample including nucleic acid-containing material and inhibitors; transferring the lysed sample to the separation chamber to contact the solid phase material and to preferentially adhere at least a portion of the inhibitors to the solid phase material; wherein the solid phase material includes capture sites (e.g., chelating functional groups), a coating reagent coated on the solid phase material, or both; wherein the coating reagent is selected from the group consisting of a surfactant, a strong base, a polyelectrolyte, a selectively permeable polymeric barrier, and combinations thereof; and separating at least a portion of the nucleic acid-containing material from the solid phase material having at least a portion of the inhibitors adhered thereto; and after separating the nucleic acid-containing material, further lysing the nucleic acid-containing material to release nucleic acid.

As discussed above with respect to Illustrative Method 1, the nucleic acid-containing material and cells containing inhibitors of the embodiments of Illustrative Method 2 may be the same or different, although they are typically different. That is, the nucleic acid containing material and the inhibitor-containing cells could potentially be the same. Also, in illustrative Method 2, the sample can include free nucleic acid and free inhibitors.

In certain embodiments of such methods, separating the nucleic acid-containing material (e.g., nuclei) (and/or nucleic acid if already isolated from the nucleic acid-containing material) from the solid phase material includes transferring the nucleic acid-containing material (e.g., nuclei) (and/or nucleic acid) to an amplification reaction chamber (e.g., a PCR sample chamber).

In certain embodiments of such methods, the solid phase material includes a polytetrafluoroethylene fibril matrix, sorptive particles enmeshed in the matrix, and a coating reagent coated on the solid phase material, wherein the coating reagent is selected from the group consisting of a surfactant, a strong base, a polyelectrolyte, a selectively permeable polymeric barrier, and combinations thereof.

In certain embodiments of such methods, separating includes eluting at least a portion of the nucleic acid-containing material (e.g., nuclei) (and/or nucleic acid) from the solid phase material with an eluting reagent.

Referring to FIG. 2, a preferred embodiment of the microfluidic device suitable for use with these embodiments includes a loading chamber 30, an optional mixing chamber 32, a separation chamber 36 that includes a solid phase material, an eluting reagent chamber 38, and an optional amplification chamber 42. These chambers are in fluid communication with each other such that a sample can be loaded into the loading chamber 30, which can then be transferred to the mixing chamber 32, or if it is not present, directly to the separation chamber 36. If the sample is premixed with a lysing reagent (i.e., pre-lysed), for example, then the mixing chamber 32 may not be needed. Alternatively, the mixing chamber 32 could include a lysing reagent, for example, in a pre-deposited (and typically, a dried-down) form. The lysed sample is transferred to the separation chamber 36 for contact with the solid phase material to preferentially adhere at least a portion of the inhibitors to the solid phase material. The eluting reagent in the eluting reagent chamber 38 is then transferred to the separation chamber 36 to remove at least a portion of the target nucleic acid-containing material and/or nucleic acid. The sample can be eluted with a strong base for further lysing. After lysis using a strong base, in some cases, the samples can be heated for a few minutes at high temperatures (95° C.) to denature proteins that are inhibitory to PCR followed by neutralization using an agent such as TRIS buffer to adjust the pH of the sample prior to PCR. Alternatively, the eluted sample can be transferred to a chamber for further lysing using other methods (e.g., heating, Proteinase K). Alternatively, these reagents could be pre-deposited (and dried-down) if desired. In certain embodiments, this material can be directly transferred to an amplification reaction chamber 42 for carrying out a PCR process, for example. The amplification reaction chamber 42 can optionally include pre-deposited reactants for the amplification reaction (e.g., PCR).

### Additional Embodiments

In certain embodiments of such methods that involve the use of a microfluidic device, placing the sample in the loading chamber occurs prior to contacting the sample with a first lysing reagent. Alternatively, placing the sample in the loading chamber occurs after contacting the sample with a first lysing reagent. The first lysing reagent can be water or a nonionic surfactant, for example.

If additional lysing is needed to release nucleic acid from nucleic acid-containing material (e.g., nuclei), other lysing conditions can be used. For example, this includes subjecting the nucleic acid-containing material to a strong base with optional heating. The strong base is typically NaOH, but can be others such as KOH, LiOH, NH₄OH, as well as primary, secondary, or tertiary amines. Typically, the temperature is at room temperature. If a base is used, the sample containing the released nucleic acid may need to have its pH adjusted, particularly if the nucleic acid is to be subjected to a subsequent amplification process. Thus, certain embodiments of the invention include adjusting the pH of the sample typically to at least 7.5, and typically to no greater than 9.

In certain embodiments of such methods that involve the use of a microfluidic device, the first lysing reagent is a nonionic surfactant. In certain embodiments of such methods, the loading chamber includes the first lysing reagent (e.g., pre-deposited (and typically, dried-down) nonionic surfactant) and contacting the sample with a first lysing reagent occurs upon placing the sample in the loading chamber.

Alternatively, the sample could be transferred to a subsequent processing chamber with the first lysing reagent (preferably, a nonionic surfactant) therein. For example, contacting the sample with a first lysing reagent (preferably, a nonionic surfactant) occurs in a mixing chamber with sufficient mixing to break cell membranes and release nuclei and inhibitors to form a lysed sample. It should be understood that if a “first” lysing reagent is used, the methods do not necessarily require the use of a “second” lysing reagent; rather, the term “first lysing reagent” is used herein to distinguish from any additional lysing reagents if used.

As stated above, the addition of sucrose in a buffer (particularly, a TRIS buffer) may help in the isolation of nuclei. The buffer could also include magnesium salts and surfactants such as TRITON X-100. This may also provide a good medium for lysis of white blood cells. Furthermore, in certain cases, when the nuclei need to be archived, particularly within a microfluidic device, using a nuclei storage buffer may be useful. The nuclei storage buffer could include sucrose, magnesium salts, EDTA, dithiothrietol, 4-(2-aminoethyl)benzenesulfonyl fluoride (AEBSF), and/or glycerol, for example, in a buffer (e.g., TRIS buffer) and would allow for stable storage of nuclei.

In certain embodiments of such methods that involve the use of a microfluidic device, forming a concentrated region of the sample in the valved process chamber includes centrifuging the sample in the process chamber. Typically, separating the concentrated region from the less concentrated of the sample includes transferring the less concentrated region of the sample through the valve to a waste bin. Spinning speeds, such as 400 rcf for 2 minutes, are typically desirable when using a microfluidic device, although higher spinning speeds and/or longer spinning times may be used.

The nonionic surfactant, such as TRITON X-100, can be pre-deposited (and dried down uniformly if desired) in a mixing chamber of a microfluidic device, for example, such that lysing occurs when the sample (e.g., blood) is mixed with the surfactant for a few minutes. In other cases, a dilute solution of surfactant can be pre-mixed with the sample (e.g., blood) prior to introduction into the mixing chamber.

In other cases, when a nonionic surfactant is not used, water or ammonium chloride can be used for lysis of red blood cells. After lysis occurs in the mixing chamber, the white blood cells can be centrifuged down at relatively low speeds.

In certain embodiments of the methods described herein, the sample can be whole blood. The whole blood is then typically separated into component parts and the portion containing white blood cells (typically referred to as the buffy coat) separated and lysed to release the nuclei and/or nucleic acid. For example, in certain embodiments, the method can include centrifuiging the whole blood (e.g., in a valved process chamber) to form a plasma layer (often in the upper layer), a red blood cell layer (often in the lower layer), and an interfacial layer that includes white blood cells, and removing a substantial portion of the interfacial layer (i.e., buffy coat). The buffy coat can then be subjected to further processing.

In certain embodiments, the buffy coat could be separated from whole blood using conventional techniques. The buffy coat could then be used as the sample in the methods described herein.

In addition to the solid phase material discussed herein, other types of solid phase materials, particularly beads, can be introduced into a microfluidic device in a variety of embodiments of the present invention. For example, beads can be functionalized with the appropriate groups to isolate specific cells, viruses, bacteria, proteins, nucleic acids, etc. The beads can be segregated from the sample by centrifugation and subsequent separation. The beads could be designed to have the appropriate density and sizes (nanometers to microns) for segregation. For example, in the case of viral capture, beads that recognize the protein coat of a virus can be used to capture and concentrate the virus prior to or after removal of small amounts of residual inhibitors from a serum sample.

The inhibitors can be removed using solid phase materials, as described herein, prior to or after capture of viral particles onto the beads. In addition to this, the amount of inhibitors can be reduced using concentration/separation/optional dilution steps, for example, as disclosed in U.S. patent application Ser. No. ______, filed on ______, entitled METHODS FOR NUCLEIC ACID ISOLATION AND KITS USING A MICROFLUIDIC DEVICE AND CONCENTRATION STEP (Attorney Docket No. 59801US002). Such concentration/separation/optional dilution steps can be used with various methods and samples described herein.

Nucleic acids can be extracted out of the segregated viral particles by lysis. Thus, the beads could provide a way of concentrating relevant material in a specific region within a microfluidic device, also allowing for washing of irrelevant materials and elution of relevant material from the captured particle.

Examples of such beads include, but are not limited to, crosslinked polystyrene beads available under the trade designation CHELEX from Bio-Rad Laboratories, Inc. (Hercules, Calif.), crosslinked agarose beads with tris(2-aminoethyl)amine, iminodiacetic acid, nitrilotriacetic acid, polyamines and polyimines as well as the chelating ion exchange resins commercially available under the trade designation DUOLITE C-467 and DUOLITE GT73 from Rohm and Haas (Philadelphia, Pa.), AMBERLITE IRC-748, DIAION CR11, DUOLITE C647. These beads are also suitable for use as the solid phase material as discussed above.

Other examples of beads include those available under the trade designations GENE FIZZ (Eurobio, France), GENE RELEASER (Bioventures Inc., Murfreesboro, Tenn.), and BUGS N BEADS (GenPoint, Oslo, Norway), as well as Zymo's beads (Zymo Research, Orange, Calif.) and DYNAL beads (Dynal, Oslo, Norway).

Other materials are also available for pathogen capture (e.g., viral particles, bacteria). For example, polymer coatings can also be used to isolate specific cells, viruses, bacteria, proteins, nucleic acids, etc. in certain embodiments of the invention. These polymer coatings could directly be spray-jetted, for example, onto the cover film of a microfluidic device.

Viral particles can be captured onto beads by covalently attaching antibodies onto bead surfaces. The antibodies can be raised against the viral coat proteins. For example, DYNAL beads can be used to covalently link antibodies. Alternatively, synthetic polymers, for example, anion-exchange polymers, can be used to concentrate viral particles. Commercially available resins such as viraffinity (Biotech Support Group, East Brunswick, N.J.) can be used to coat beads or applied as polymer coatings onto select locations in microfluidic device to concentrate viral particles. BUGS N BEADS (GenPoint, Oslo, Norway) can, for example, be used for extraction of bacteria. Here, these beads can be used to capture bacteria such as Staphylococcus, Streptococcus, E coli, Salmonella, and Clamydia elementary bodies.

Thus, in one embodiment of the present invention when the sample includes viral particles or other pathogens (e.g., bacteria), a microfluidic device can include solid phase material in the form of viral capture beads or other pathogen capture material. More specifically, in one case, the beads can be used only for concentration of virus or bacteria, for example, followed by segregation of beads to another chamber, ending with lysis of virus or bacteria. In another case, the beads can be used for concentration of virus or bacteria, followed by lysis and capture of nucleic acids onto the same bead, dilution of beads, concentration of beads, segregation of beads, and repeating the process multiple times prior to elution of captured nucleic acid.

If the downstream application of the nucleic acid is subjecting it to an amplification process such as PCR, then all reagents used in the method are preferably compatible with such process (e.g., PCR compatible). Furthermore, the addition of PCR facilitators may be useful, especially for diagnostic purposes. Also, heating of the material to be amplified prior to amplification can be beneficial.

In embodiments in which the inhibitors are not completely removed, the use of buffers, enzymes, and PCR facilitators can be added that help in the amplification process in the presence of inhibitors. For example, enzymes other than Taq Polymerase, such as rTth, that are more resistant to inhibitors can be used, thereby providing a huge benefit for PCR amplification. The addition of Bovine Serum Albumin, betaine, proteinase inhibitors, bovine transferrin, etc. can be used as they are known to help even further in the amplification process. Alternatively, one can use a commercially available product such as Novagen's Blood Direct PCR Buffer kit (EMD Biosciences, Darmstadt, Germany) for direct amplification from whole blood without the need for extensive purification.

Objects and advantages of this invention are further illustrated by the following examples, but the particular materials and amounts thereof recited in these examples, as well as other conditions and details, should not be construed to unduly limit this invention.

### EXAMPLES

### Example 1A

### Preparation of Solid Phase Material: Ammonia Form without TRITON X-100

A 3M No. 2271 EMPORE Extraction Chelating Disk (3M, St. Paul, Minn.) was placed in a glass filter holder. The extraction disk was converted into the ammonia form, following the procedure printed on the package insert.

### Example 1B

### Preparation of Solid Phase Material: Ammonia Form with TRITON X-100

A 3M No. 2271 EMPORE Extraction Chelating Disk was placed in a glass filter holder. The extraction disk was converted into the ammonia form, following the procedure printed on the package insert. The disk placed in a vial and was submerged in a 1% TRITON X-100 (Sigma-Aldrich, St. Louis, Mo.) solution (0.1 gram (g) of TRITON X-100 in 10 mL of water), mixing for about 6-8 hours on a Thermolyne Vari-Mix Model M48725 Rocker (Bamstead/Thermolyne, Dubuque, Iowa). The disk was placed in glass filter holder, dried by applying a vacuum for about 20 minutes (min), and then dried overnight at room temperature (approximately 21° C.), taking care not to wash or rinse the disk.

### Example 1C

### Preparation of Solid Phase Material: Anion-SR Empore Chelating Membrane with TRITON X-100

A 3M No. 2252 EMPORE Extraction Chelating Disk was placed in a vial and was submerged in a 1% TRITON X-100 solution (0.1 g of TRITON X-100 in 10 milliliters (mL) of water), mixing for about 6-8 hours on a Thermolyne Vari-Mix Model M48725 Rocker. The disk was placed in glass filter holder, dried by applying a vacuum for about 20 minutes, and then dried overnight at room temperature (approximately 21° C.), taking care not to wash or rinse the disk.

### Example 1D

### Preparation of Solid Phase Material: C8 Chelating Membrane with TRITON X-100

A 3M No. 2214 EMPORE Extraction Chelating Disk was placed in a vial and was submerged in a 1% TRITON X-100 solution (0.1 g of TRITON X-100 in 10 mL of water), mixing for about 6-8 hours on a Thermolyne Vari-Mix Model M48725 Rocker. The disk was placed in glass filter holder, dried by applying a vacuum for about 20 minutes, and then dried overnight at room temperature (approximately 21° C.), taking care not to wash or rinse the disk.

### Example 1E

### Preparation of Solid Phase Material: Chelating Membrane with TRITON X-100 and FR-2025

A 3M No. 2271 EMPORE Extraction Chelating Disk was placed in a vial and was submerged in a 50/50 mixture of 1% TRITON X-100 (0.1 g of TRITON X-100 in 10 milliliters (mL) of water) and 0.1% FR-2025 (3M, St. Paul, Minn.) and mixed for about 6-8 hours on a Thermolyne Vari-Mix Model M48725 Rocker. The disk was placed in glass filter holder, dried by applying a vacuum for about 20 minutes, and then dried overnight at room temperature (approximately 21° C.), taking care not to wash or rinse the disk.

### Example 1F

### Preparation of Solid Phase Material: Chelating Membrane with TRITON X-100 and FC-4430

A 3M No. 2271 EMPORE Extraction Chelating Disk was placed in a vial and was submerged in a 50/50 mixture of 1% TRITON X-100 (0.1 g of TRITON X-100 in 10 milliliters (mL) of water) and 0.1% FC-4430 (3M, St. Paul, Minn.) and mixed for about 6-8 hours on a Thermolyne Vari-Mix Model M48725 Rocker. The disk was placed in glass filter holder, dried by applying a vacuum for about 20 minutes, and then dried overnight at room temperature (approximately 21° C.), taking care not to wash or rinse the disk.

### Example 1G

### Preparation of Solid Phase Material: Chelating Membrane with TRITON X-100 and PSSA

A 3M No. 2271 EMPORE Extraction Chelating Disk was placed in a glass filter holder. The extraction disk was converted into the ammonia form, following the procedure printed on the package insert. The disk placed in a vial and was submerged in a 50/50 mixture of 1% TRITON X-100 (0.1 g of TRITON X-100 in 10 milliliters (mL) of water) and 1% PSSA (Poly(sodium 4-styrene-sulfonate) (Sigma-Aldrich, St. Louis, Mo.) (20 wt-% PSSA stock solution was diluted to 1 wt-% in RNAse-free sterile water) and mixed for about 6-8 hours on a Thermolyne Vari-Mix Model M48725 Rocker. The disk was placed in glass filter holder, dried by applying a vacuum for about 20 minutes, and then dried overnight at room temperature (approximately 21° C.), taking care not to wash or rinse the disk.

### Example 1H

### Preparation of Solid Phase Material: Cation-SR EMPORE Chelating Membrane with TRITON X-100

A 3M No. 2251 EMPORE Extraction Chelating Disk was placed in a vial and was submerged in a 1% TRITON X-100 solution (0.1 g of TRITON X-100 in 10 mL of water), mixing for about 6-8 hours on a Thermolyne Vari-Mix Model M48725 Rocker. The disk was placed in glass filter holder, dried by applying a vacuum for about 20 minutes, and then dried overnight at room temperature (approximately 21° C.), taking care not to wash or rinse the disk.

### Example 1J

### Preparation of Solid Phase Material: EMPORE Chelating Membrane with TRITON X-100

A 3M No. 2241 EMPORE Extraction Chelating Disk was placed in a vial and was submerged in a 1% TRITON X-100 solution (0.1 g of TRITON X-100 in 10 mL of water), mixing for about 6-8 hours on a Thermolyne Vari-Mix Model M48725 Rocker. The disk was placed in glass filter holder, dried by applying a vacuum for about 20 minutes, and then dried overnight at room temperature (approximately 21° C.), taking care not to wash or rinse the disk.

### Example 2

### Procedure for Isolation of Genomic DNA from Whole Blood Using Chelating Solid Phase Material

One (1) μL of neat TRITON X-100 was added to one hundred (100) μL of whole blood. The solution was incubated at room temperature (approximately 21° C.) for about 5 minutes, vortexing the solution intermittently (for approximately 5 seconds every 20 seconds). The solution was investigated to make sure that it was transparent before proceeding to the next step. The solution was spun in an Eppendorf Model 5415 D centrifuge (Brinkmann, Westbury, N.Y.) at 400 rcf for about 10 minutes. The supernatant was separated and discarded, leaving about two (2) μL of concentrated material at the bottom of the centrifuge tube. This material was transferred to a 3M No. 2271 EMPORE Extraction Chelating Disk, making sure that the material was evenly distributed on the surface of the disk. The material was allowed to dry on the disk for about 2-5 minutes until the intense red color became darker. Twelve microliters (12 μL) of 0.083 molar (M) NaOH was added to the extraction disk. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. A 2 μL aliquot was removed and added to 10 μL of 40 millimolar (mM) TRIS-HCl (Sigma-Aldrich, St. Louis, Mo.) (pH 7.4).

### Example 3

### Procedure for Isolation of Genomic DNA from Whole Blood Using Ammoniated Chelating Solid Phase Material

One (1) μL of neat TRITON X-100 was added to one hundred (100) μL of whole blood. The solution was incubated at room temperature (approximately 21° C.) for about 5 minutes, vortexing the solution intermittently (for approximately 5 seconds every 20 seconds). The solution was investigated to make sure that it was transparent before proceeding to the next step. The solution was spun in an Eppendorf Model 5415D centrifuge at 400 rcf for about 10 minutes. The supernatant was separated and discarded, leaving about two (2) μL of concentrated material at the bottom of the centrifuge tube. This material was transferred to the extraction membrane prepared in Example 1A, making sure that the material was evenly distributed on the surface of the membrane. The material was allowed to dry on the membrane for about 2-5 minutes until the intense red color became darker. Twelve (12) μL of 0.083 M NaOH was added to the extraction membrane. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. A 2 μL aliquot was removed and added to 10 μL of 40 mM TRIS-HCl (pH 7.4).

### Example 4

### Procedure for Isolation of Genomic DNA from Whole Blood Using Ammoniated Chelating Solid Phase Material with TRITON X-100

One (1) μL of neat TRITON X-100 was added to one hundred (100) μL of whole blood. The solution was incubated at room temperature (approximately 21° C.) for about 5 minutes, vortexing the solution intermittently (for approximately 5 seconds every 20 seconds). The solution was investigated to make sure that it was transparent before proceeding to the next step. The solution was spun in an Eppendorf Model 5415D centrifuge at 400 rcf for about 10 minutes. The supematant was separated and discarded, leaving about two (2) μL of concentrated material at the bottom of the centrifuge tube. This material was transferred to the extraction membrane prepared in Example 1B, making sure that the material was evenly distributed on the surface of the membrane. The material was allowed to dry on the membrane for about 2-5 minutes until the intense red color became darker. Twelve (12) μL of 0.083 M NaOH was added to the extraction membrane. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. The solution was mixed up and down 2-3 times in a pipette tip and removed after mixing. A 2 μL aliquot was removed and added to 10 μL of 40 mM TRIS-HCl (pH 7.4).

### Example 5A

### Concentration of Genomic DNA by Spinning Whole Blood Prior to Treatment with Solid Phase Material: Sample Isolation From the Top of the Tube

One (1) μL of neat TRITON X-100 was added to one hundred (100) μL of whole blood. The solution was incubated at room temperature (approximately 21° C.) for about 5 minutes, vortexing the solution intermittently (for approximately 5 seconds every 20 seconds). The solution was investigated to make sure that it was transparent before proceeding to the next step. The solution was spun in an Eppendorf Model 5415D centrifuige at 400 rcf for about 10 minutes. A two (2) μL aliquot was removed from the top of the centrifuge tube and was transferred to the extraction membrane prepared in Example 1B, making sure that the material was evenly distributed on the surface of the membrane. The material was allowed to dry on the membrane for about 2-5 minutes until the intense red color became darker. Twelve (12) μL of 0.083 M NaOH was added to the extraction membrane The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. A 2 μL aliquot was removed and added to 10 μL of 40 mM TRIS-HCl (pH 7.4).

### Example 5B

### Concentration of Genomic DNA by Spinning Whole Blood Prior to Treatment with Solid Phase Material: Sample Isolation from the Bottom of the Tube

One (1) μL of neat TRITON X-100 was added to one hundred (100) μL of whole blood. The solution was incubated at room temperature (approximately 21° C.) for about 5 minutes, vortexing the solution intermittently (for approximately 5 seconds every 20 seconds). The solution was investigated to make sure that it was transparent before proceeding to the next step. The solution was spun in an Eppendorf Model 5415D centrifuge at 400 rcf for about 10 minutes. A two (2) μL aliquot was removed from the bottom of the centrifuge tube and was transferred to the extraction membrane prepared in Example 1B, making sure that the material was evenly distributed on the surface of the membrane. The material was allowed to dry on the membrane for about 2-5 minutes until the intense red color became darker. Twelve (12) μL of 0.083 M NaOH was added to the extraction membrane. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. A 2 μL aliquot was removed and added to 10 μL of 40 mM TRIS-HCl (pH 7.4).

### Example 5C

### Purification of Genomic DNA by Treatment with Solid Phase Material: Sample Isolation from the Top of the Tube—No Concentration Step

One (1) μL of neat TRITON X-100 was added to one hundred (100) μL of whole blood. The solution was incubated at room temperature (approximately 21° C.) for about 5 minutes, vortexing the solution intermittently (for approximately 5 seconds every 20 seconds). The solution was investigated to make sure that it was transparent before proceeding to the next step. A two (2) μL aliquot was removed from the top of the centrifuge tube and was transferred to the extraction membrane prepared in Example 1B, making sure that the material was evenly distributed on the surface of the membrane. The material was allowed to dry on the membrane for about 2-5 minutes until the intense red color became darker. Twelve (12) μL of 0.083 M NaOH was added to the extraction membrane. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. A 2 μL aliquot was removed and added to 10 μL of 40 mM TRIS-HCl (pH 7.4).

### Example 6A

### Concentration of Genomic DNA by Spinning Whole Blood Followed by QIAamp Clean-up: Sample Isolation from the Top of the Tube

One (1) μL of neat TRITON X-100 was added to one hundred (100) μL of whole blood. The solution was incubated at room temperature (approximately 21° C.) for about 5 minutes, vortexing the solution intermittently (for approximately 5 seconds every 20 seconds). The solution was investigated to make sure that it was transparent before proceeding to the next step. The solution was spun in an Eppendorf Model 5415D centrifuge at 400 rcf for about 10 minutes. A two (2) μL aliquot was removed from the top of the centrifuige tube and was added to 198 μL of 1×Phosphate buffer saline (PBS). Clean DNA was obtained, following “Blood and Body Fluid Spin Protocol” described in QIAamp DNA Blood Mini Kit Handbook p. 27(Qiagen, Valencia, Calif.), eluting in 72 μL of water.

### Example 6B

### Concentration of Genomic DNA by Spinning Whole Blood Followed by QIAamp Clean-up: Sample Isolation from the Bottom of the Tube

One (1) μL of neat TRITON X-100 was added to one hundred (100) μL of whole blood. The solution was incubated at room temperature (approximately 21° C.) for about 5 minutes, vortexing the solution intermittently (for approximately 5 seconds every 20 seconds). The solution was investigated to make sure that it was transparent before proceeding to the next step. The solution was spun in an Eppendorf Model 5415D centrifuge at 400 rcf for about 10 minutes. A two (2) μL aliquot was removed from the bottom of the centrifuge tube and was added to 198 μL of 1×Phosphate buffer saline (PBS). Clean DNA was obtained, following “Blood and Body Fluid Spin Protocol” described in QIAamp DNA Blood Mini Kit Handbook p. 27, eluting in 72 μL of water.

### Example 6C

### Collecttion of Genomic DNA by QIAamp Clean-up: Sample Isolation from the Top of the Tube—No Centrifuging

One (1) μL of neat TRITON X-100 was added to one hundred (100) μL of whole blood. The solution was incubated at room temperature (approximately 21° C.) for about 5 minutes, vortexing the solution intermittently (for approximately 5 seconds every 20 seconds). The solution was investigated to make sure that it was transparent before proceeding to the next step. A two (2) μL aliquot was removed from the top of the centrifuge tube and was added to 198 μL of 1×Phosphate buffer saline (PBS, Sigma-Aldrich, St. Louis, Mo.). Clean DNA was obtained, following “Blood and Body Fluid Spin Protocol” described in QIAamp DNA Blood Mini Kit Handbook p. 27, eluting in 72 μL of water.

### Example 7A

### Effect of Inhibitor/DNA on PCR: Varying Inhibitor Concentration with Fixed DNA Concentration

A dilution series of inhibitors were made prior to spiking with clean human genomic DNA in order to study the effect of inhibitor on PCR. To 10 μL of 15 nanograms per microliter (ng/μL) human genomic DNA, 1 μL of different Mix I (neat or dilutions thereof) was added (Samples 7—no inhibitor added, 7D—neat, 7E—1:10, 7F—1:30, 7G—1:100, 7H—1:300) and vortexed. Two (2) μL aliquots of each sample were taken for 20 μL PCR. The results are shown in Table 2.

Mix I: one hundred (100) μL of whole blood was added to 1 μL of neat TRITON X-100. The solution was incubated at room temperature (approximately 21° C.) for about 5 minutes, vortexing the solution intermittently (for approximately 5 seconds every 20 seconds). The solution was investigated to make sure that it was transparent before proceeding to the next step. The solution was spun in an Eppendorf Model 5415D centrifuge at 400 rcf for about 10 minutes. Approximately 80 μL from the top of the microcentrifuge tube and designated Mix I.

### Example 7B

### Effect of Inhibitor/DNA on PCR: Varying DNA Concentration with Fixed Inhibitor Concentration

To 10 μL of human genomic DNA, 1 μL of 1:3 diluted Mix I (described above) was added. DNA concentrations that were examined were the following: Samples 7J—15 ng/μL, 7K—7.5 ng/μL, 7L—3.75 ng/μL, 7M—1.5 ng/μL. Two (2) μL aliquots of each sample were taken for 20 μL PCR. The results are shown in Table 2.

### Example 7C

### Effect of Inhibitor/DNA on PCR: DNA with No Added Inhibitor

The following samples were prepared with 1 μL of water added to each DNA sample instead of inhibitor: Samples 7N—15 ng/μL, 7P—7.5 ng/μL, 7Q—3.75 ng/μL, 7R—1.5 ng/μL. Two (2) μL aliquots of each sample were taken for 20 μL PCR. The results are shown in Table 2.

TABLE 2

| Sample | Ct (duplicate |
|---|---|
| No. | samples) |
| 7 | 19.10 |
| | 19.06 |
| 7D | 13.94 |
| | 29.50 |
| 7E | 27.39 |
| | 26.22 |
| 7F | 21.44 |
| | 20.66 |
| 7G | 19.90 |
| | 19.30 |
| 7H | 19.90 |
| | 20.08 |
| 7J | 28.45 |
| | 28.61 |
| 7K | 29.16 |
| | 30.22 |
| 7L | 30.47 |
| | 29.96 |
| 7M | 28.43 |
| | 26.16 |
| 7N | 20.05 |
| | 19.80 |
| 7P | 20.74 |
| | 20.54 |
| 7Q | 21.95 |
| | 21.88 |
| 7R | 22.67 |
| | 23.10 |

### Example 8A

### Recovery of Nuclei from Solid Phase Material

One (1) μL of neat TRITON X-100 was added to 2 μL of white blood cells (PBMCs). The solution was vortexed briefly, and was spun in an Eppendorf Model 5415D centrifuge at 400 rcf for about 1 minute. A three (3) μL was placed on a chelating membrane prepared as in Example 1B. The material was allowed to dry on the membrane for about 2-5 minutes. Thirteen (13) μL of 0.077 M NaOH was added to the extraction membrane. The solution was mixed up and down 2-3 times in a pipette tip and removed after mixing (although less than 13 μL might be obtained). If the solution was foamy, it was spun down at 4,000 revolutions per minute (rpm) for 1 minute. A 2 μL aliquot was removed and added to 10 μL of 40 mM TRIS-HCl (pH 7.4).

### Example 8B

### Recovery of Nuclei with No Solid Phase Material

One (1) μL of neat TRITON X-100 was added to 2 μL of white blood cells (PBMCs). The solution was vortexed briefly, and was spun in an Eppendorf Model 5415D centrifuge at 400 rcf for about 1 minute. Ten (10) μL of 0.1 M NaOH was added to the solution and incubated at room temperature (approximately 21° C.) for about 5 minutes. A 2 μL aliquot was removed and added to 10 μL of 40 mM TRIS-HCl (pH 7.4).

### Example 8C

### Sample Spiked Afterwards with Nuclei

One (1) μL of neat TRITON X-100 was added to 2 μL of water. The solution was vortexed briefly, and was spun in an Eppendorf Model 5415D centrifuge at 400 rcf for about 1 minute. A three (3) μL was placed on a chelating membrane prepared as in Example 1B. The material was allowed to dry on the membrane for about 2-5 minutes. Thirteen (13) μL of 0.077 M NaOH was added to the extraction membrane. The solution was mixed up and down 2-3 times in a pipette tip and removed after mixing (although less than 13 μL might be obtained). If the solution was foamy, it was spun down at 4,000 revolutions per minute (rpm) for 1 minute. Two (2) μL of white blood cells (PBMCs) was added to a 10 μL aliquot of the solution and incubated at room temperature (approximately 21° C.) for about 5 minutes. A 2 μL aliquot was removed and added to 10 μL of 40 mM TRIS-HCl (pH 7.4).

### Example 9A

### Procedure for Isolation of Genomic DNA from Whole Blood Using Solid Phase Material: Extraction from the Solid Phase Material with NaOH

One (1) μL of neat TRITON X-100 was added to one hundred (100) μL of whole blood. The solution was incubated at room temperature (approximately 21° C.) for about 5 minutes, vortexing the solution intermittently (approximately 5 seconds every 20 seconds). The solution was investigated to make sure that it was transparent before proceeding to the next step. The solution was spun in an Eppendorf Model 5415D centrifuge at 400 rcf for about 10 minutes. The supernatant was separated and discarded, leaving about two (2) μL of concentrated material at the bottom of the centrifuge tube. This material was transferred to the extraction membrane prepared in Example 1B, making sure that the material was evenly distributed on the surface of the membrane. The material was allowed to dry on the membrane for about 2-5 minutes until the intense red color became darker. Twelve (12) μL of 0.083 M NaOH was added to the extraction membrane. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. Fifty (50) μL of 1×TE buffer (Sigma-Aldrich, St. Louis, Mo.) was added to a 10 μL aliquot of the solution. Three hundred (300) μL of 40 mM TRIS-HCl (pH 7.4) was added to the solution.

### Example 9B

### Procedure for Isolation of Genomic DNA from Whole Blood Using Solid Phase Material: Extraction from the Solid Phase Material with Water

One (1) μL of neat TRITON X-100 was added to one hundred (100) μL of whole blood. The solution was incubated at room temperature (approximately 21° C.) for about 5 minutes, vortexing the solution intermittently (approximately 5 seconds every 20 seconds). The solution was investigated to make sure that it was transparent before proceeding to the next step. The solution was spun in an Eppendorf Model 5415D centrifuge at 400 rcf for about 10 minutes. The supernatant was separated and discarded, leaving about two (2) μL of concentrated material at the bottom of the centrifuge tube. This material was transferred to the extraction membrane prepared in Example 1B, making sure that the material was evenly distributed on the surface of the membrane. The material was allowed to dry on the membrane for about 2-5 minutes until the intense red color became darker. Twelve (12) μL of water was added to the extraction membrane. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. Fifty (50) μL of 0.1 M NaOH was added to a 10 μL aliquot of the solution and incubated at room temperature (approximately 21° C.) for about 5 minutes. Three hundred (300) μL of 40 mM TRIS-HCl (pH 7.4) was added to the solution.

### Example 10

### Genomic DNA Isolated from Whole Blood on Ammoniated Chelating Solid Phase Material Treated with TRITON X-100

One (1) μL of 3% TRITON X-100 was added to two (2) μL of whole blood. The solution was incubated at room temperature (approximately 21° C.) for about 5 minutes, vortexing the solution intermittently (approximately 5 seconds every 20 seconds). The solution was investigated to make sure that it was transparent before proceeding to the next step. This material was transferred to the extraction membrane prepared in Example 1B, making sure that the material was evenly distributed on the surface of the membrane. The material was allowed to dry on the membrane for about 2-5 minutes until the intense red color became darker. Thirteen (13) μL of 0.083 M NaOH was added to the extraction membrane. The solution was mixed up and down 2-3 times in a pipette tip and removed after mixing. A 2 μL aliquot was removed and added to 10 μL of 40 mM TRIS-HCl (pH 7.4).

### Example 11

### Procedure for Isolation of Genomic DNA from Whole Blood Using Anion-SR Chelating Solid Phase Material

One (1) μL of neat TRITON X-100 was added to one hundred (100) μL of whole blood. The solution was incubated at room temperature (approximately 21° C.) for about 5 minutes, vortexing the solution intermittently (approximately 5 seconds every 20 seconds). The solution was investigated to make sure that it was transparent before proceeding to the next step. The solution was spun in an Eppendorf Model 5415 D centrifuge at 400 rcf for about 10 minutes. The supernatant was separated and discarded, leaving about two (2) μL of concentrated material at the bottom of the centrifuge tube. This material was transferred to a 3M No. 2252 EMPORE Anion-SR Extraction Chelating Disk prepared as described in Example 1C, making sure that the material was evenly distributed on the surface of the disk. The material was allowed to dry on the disk for about 2-5 minutes until the intense red color became darker. Twelve (12) μL of 0.083 M NaOH was added to the extraction disk. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. A 2 μL aliquot was removed and added to 10 μL of 40 mM TRIS-HCl (pH 7.4).

### Example 12

### Procedure for Isolation of Genomic DNA from Whole Blood Using C8 Chelating Solid Phase Material

One (1) μL of neat TRITON X-100 was added to one hundred (100) μL of whole blood. The solution was incubated at room temperature (approximately 21° C.) for about 5 minutes, vortexing the solution intermittently (approximately 5 seconds every 20 seconds). The solution was investigated to make sure that it was transparent before proceeding to the next step. The solution was spun in an Eppendorf Model 5415 D centrifuge at 400 rcf for about 10 minutes. The supernatant was separated and discarded, leaving about two (2) μL of concentrated material at the bottom of the centrifuge tube. This material was transferred to a 3M No. 2214 EMPORE. C-8 Extraction Chelating Disk prepared as described in Example 1D, making sure that the material was evenly distributed on the surface of the disk. The material was allowed to dry on the disk for about 2-5 minutes until the intense red color became darker. Twelve (12) μL of 0.083 M NaOH was added to the extraction disk. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. A 2 μL aliquot was removed and added to 10 μL of 40 mM TRIS-HCl (pH 7.4).

### Example 13

### Procedure for Isolation of Genomic DNA from Whole Blood Using Chelating Solid Phase Material

One (1) μL of neat TRITON X-100 was added to one hundred (100) μL of whole blood. The solution was incubated at room temperature (approximately 21° C.) for about 5 minutes, vortexing the solution intermittently (approximately 5 seconds every 20 seconds). The solution was investigated to make sure that it was transparent before proceeding to the next step. The solution was spun in an Eppendorf Model 5415 D centrifuge at 400 rcf for about 10 minutes. The supernatant was separated and discarded, leaving about two (2) μL of concentrated material at the bottom of the centrifuge tube. This material was transferred to a 3M No. 2271 EMPORE Extraction Chelating Disk prepared as described in Example 1E, making sure that the material was evenly distributed on the surface of the disk. The material was allowed to dry on the disk for about 2-5 minutes until the intense red color became darker. Twelve (12) μL of 0.083 M NaOH was added to the extraction disk. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. A 2 μL aliquot was removed and added to 10 μL of 40 mM TRIS-HCl (pH 7.4).

### Example 14

### Procedure for Isolation of Genomic DNA from Whole Blood Using Chelating Solid Phase Material

One (1) μL of neat TRITON X-100 was added to one hundred (100) μL of whole blood. The solution was incubated at room temperature (approximately 21° C.) for about 5 minutes, vortexing the solution intermittently (approximately 5 seconds every 20 seconds). The solution was investigated to make sure that it was transparent before proceeding to the next step. The solution was spun in an Eppendorf Model 5415 D centrifuge at 400 rcf for about 10 minutes. The supematant was separated and discarded, leaving about two (2) μL of concentrated material at the bottom of the centrifuge tube. This material was transferred to a 3M No. 2271 EMPORE Extraction Chelating Disk prepared as described in Example 1F, making sure that the material was evenly distributed on the surface of the disk. The material was allowed to dry on the disk for about 2-5 minutes until the intense red color became darker. Twelve (12) μL of 0.083 M NaOH was added to the extraction disk. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. A 2 μL aliquot was removed and added to 10 μL of 40 mM TRIS-HCl (pH 7.4).

### Example 15

### Procedure for Isolation of Genomic DNA from Whole Blood Using Chelating Solid Phase Material

One (1) μL of neat TRITON X-100 was added to one hundred (100) μL of whole blood. The solution was incubated at room temperature (approximately 21° C.) for about 5 minutes, vortexing the solution intermittently (approximately 5 seconds every 20 seconds). The solution was investigated to make sure that it was transparent before proceeding to the next step. The solution was spun in an Eppendorf Model 5415 D centrifuge at 400 rcf for about 10 minutes. The supernatant was separated and discarded, leaving about two (2) μL of concentrated material at the bottom of the centrifuge tube. This material was transferred to a 3M No. 2271 EMPORE Extraction Chelating Disk prepared as described in Example 1G, making sure that the material was evenly distributed on the surface of the disk. The material was allowed to dry on the disk for about 2-5 minutes until the intense red color became darker. Twelve (12) μL of 0.083 M NaOH was added to the extraction disk. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. A 2 μL aliquot was removed and added to 10 μL of 40 mM TRIS-HCl (pH 7.4).

### Example 16

### Procedure for Isolation of Genomic DNA from Whole Blood Using Chelating Solid Phase Material

One (1) μL of neat TRITON X-100 was added to one hundred (100) μL of whole blood. The solution was incubated at room temperature (approximately 21° C.) for about 5 minutes, vortexing the solution intermittently (approximately 5 seconds every 20 seconds). The solution was investigated to make sure that it was transparent before proceeding to the next step. The solution was spun in an Eppendorf Model 5415 D centrifuge at 400 rcf for about 10 minutes. The supernatant was separated and discarded, leaving about two (2) μL of concentrated material at the bottom of the centrifuge tube. This material was transferred to a 3M No. 2271 EMPORE Extraction Chelating Disk prepared as described in Example 1H, making sure that the material was evenly distributed on the surface of the disk. The material was allowed to dry on the disk for about 2-5 minutes until the intense red color became darker. Twelve (12) μL of 0.083 M NaOH was added to the extraction disk. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. A 2 μL aliquot was removed and added to 10 μL of 40 mM TRIS-HCl (pH 7.4).

### Example 17

### Procedure for Isolation of Genomic DNA from Whole Blood Using Chelating Solid Phase Material

One (1) μL of neat TRITON X-100 was added to one hundred (100) μL of whole blood. The solution was incubated at room temperature (approximately 21° C.) for about 5 minutes, vortexing the solution intermittently (approximately 5 seconds every 20 seconds). The solution was investigated to make sure that it was transparent before proceeding to the next step. The solution was spun in an Eppendorf Model 5415 D centrifuge at 400 rcf for about 10 minutes. The supernatant was separated and discarded, leaving about two (2) μL of concentrated material at the bottom of the centrifuge tube. This material was transferred to a 3M No. 2271 EMPORE Extraction Chelating Disk prepared as described in Example 1J, making sure that the material was evenly distributed on the surface of the disk. The material was allowed to dry on the disk for about 2-5 minutes until the intense red color became darker. Twelve (12) μL of 0.083 M NaOH was added to the extraction disk. The solution was mixed up and down 2-3 times in a pipette tip and removed after mixing (although less than 12 μL might be obtained). The color of the sample removed from the membrane varied from colorless to faint green. If the solution was foamy, it was spun down at 4,000 revolutions per minute (rpm) for 1 minute. A 2 μL aliquot was removed and added to 10 μL of 40 mM TRIS-HCl (pH 7.4).

### Example 18

### Procedure for Isolation of Genomic DNA from Whole Blood Using Ammoniated Chelating Solid Phase Material with TRITON X-100 Mounted on a Microfluidic Device

Two (2) μL of neat TRITON X-100 was added to one hundred (100) μL of whole blood. The solution was incubated at room temperature (approximately 21° C.) for about 5 minutes, vortexing the solution intermittently (for approximately 5 seconds every 20 seconds). The solution was investigated to make sure that it was transparent before proceeding to the next step. The solution was spun in an Eppendorf Model 5415D centrifuige at 400 rcf for about 2 min. The solution from the top was discarded leaving 4 μL at the bottom of the tube. To 4 μL of concentrated material, 4 μL of RNase water was added, and the sample was mixed thoroughly. The sample then was placed into an injection port on a microfluidic device as described in Applicants' Assignee's copending U.S. patent application Ser. No. 10/734,682, filed Dec. 12, 2003 (FIG. 1). The solution was spun at 500 rpm for 5 min to a clean up chamber containing a 3M No. 2271 EMPORE Extraction Chelating Disk prepared as described in Example 1B using 10% TRITON X-100 instead of 1% TRITON X-100 as a loading solution. A five (5) μL aliquot of 0.1 M NaOH was added to a different injection port and was spun at 1000 rpm for 5 min to the clean up chamber described above. A ten (10) μL aliquot of 0.1 M NaOH was added to the same injection port, and the solution was spun down at 2500 rpm for 10 min. A 5 μL aliquot was removed and added to 1.5 μL of 1 M TRIS-HCl (pH 7.4).

### Example 19

### Procedure for Isolation of Genomic DNA from Whole Blood with the Use of a Chelating Solid Phase Material

One hundred (100) μL of whole blood was added to one hundred (100) μL 2% TRITON X-100. The solution was mixed thoroughly, and then investigated to make sure that it was transparent before proceeding to the next step. The solution was spun in an Eppendorf Model 5415D centrifuge at 400 rcf for 2 min. A 95 μL aliquot of the solution from the top was removed and discarded. Last five (5) μL containing concentrated material was placed onto a 3M No. 2271 EMPORE Extraction Chelating Disk prepared as described in Example 1B using 10% TRITON X-100 instead of 1% TRITON X-100 as a loading solution. After the solution had soaked into the disk, the sample was extracted with a twenty (20) μL aliquot of 0.1M NaOH. The solution was briefly spun in an Eppendorf Model 5415D centrifuge at 400 rcf. An aliquot of eleven (11) μL of sample was heated for 3 min at 95° C., and then added to three (3) μL of 1 M TRIS-HCl (pH 7.4).

### Example 20

### Procedure for Isolation of Genomic DNA from Whole Blood

Five (5) μL of whole blood was added to the ten (10) μL of 10 mM NaOH. After 1 min incubation, the sample was heated for 3 min at 95° C. A 5 μL aliquot of 16 mM TRIS-HCl (pH 7.4) was added to 10 μL of sample.

### Results

Table 3 reports results that were obtained on ABI 7700 QPCR Machine (Applera, Foster City, Calif.) following the instructions in QuantiTech SYBR Green PCR Handbook on p. 10-12 for preparation of a 10 μL PCR sample (2 μL of sample in 10 μL SYBR Green Master Mix, 4 μL μ-actin, 4 μL of water) for Examples 1-17, 19, and 20; Results for Example 18 were obtained on LightCycler 2.0 (Roche Applied Science, Indianapolis, Ind.) following the instructions in LightCycler Factor V Leiden Mutation Kit's package insert on p.2-3 for preparation of a 10 μL PCR sample (2.5 μL of sample in 5.5 μL of RNase-free sterile water, 1 μL of 10×Factor V Leiden Reaction Mix and 1 μL of 10×Factor V Leiden Mutation Detection Mix). One (1)% agarose gel (brightness of band—+faint, +++bright) was run on Horizon 11-14 Electrophoresis Machine (Gibco BRL, Gaithersburg, Md.). Spectra measurements were run on a SpectraMax Plus³⁸⁴ spectrophotometer at 405 nm (Molecular Devices Corporation, Sunnyvale, Calif.). Two, three or four values for each sample represent duplicates, triplicates, or quadruplicates.

TABLE 3

| Samples | Ct | Band | 405 nm (avg) |
|---|---|---|---|
| 1.5 ng/μL human | 16.92 | +++ | — |
| genomic DNA in 0.1 M | 20.67 | +++ | |
| NaOH/40 mM | | | |
| TRIS-HCl buffer | | | |
| 1.5 ng/μL human | 19.01 | +++ | 0 |
| genomic DNA in | 18.67 | +++ | |
| water | | | |
| 1.5 ng/μL human | 16.18 | +++ | — |
| genomic DNA in | 16.28 | +++ | |
| water | | | |
| Example 4 | 17-20 | +++ | 0.24 |
| | (multiple samples | +++ | |
| | analyzed) | | |
| Example 3 | 26, 28 | + | — |
| Example 2 | 28, 27 | none | — |
| Example 5A | 28 | + | 0.37 |
| | 28 | + | |
| Example 5B | 17, 18 | +++ | 0.24 |
| | | +++ | |
| Example 5C | 24 | ++ | — |
| | 26 | ++ | |
| Example 6A | 23 | + | 0 |
| | 24 | + | |
| Example 6B | 17 | +++ | 0 |
| | 18 | +++ | |
| Example 6C | 21 | ++ | 0 |
| | 21 | ++ | |
| Examples 7A and 7B | — | − | 2.63 |
| Mix I diluted 1:36 | | | |
| Examples 7A and 7B | — | − | 0.38 |
| Mix I diluted 1:360 | | | |
| Examples 7A and 7B | — | − | 0.036 |
| Mix I diluted 1:3600 | | | |
| Examples 7A and 7B | — | − | 0 |
| Mix I diluted 1:36000 | | | |
| Example 8A | 22 | +++ | |
| | 21 | | — |
| | 18 | | |
| | 22 | | |
| Example 8B | 22 | ++ | |
| | 21 | | — |
| | 18 | | |
| | 21 | | |
| Example 8C | 20 | | |
| | 20 | − | — |
| | 21 | | |
| | 22 | | |
| Example 9A | 21 | − | 0.019 |
| | 19 | | 0.007 |
| Example 9B | 25 | − | 0.029 |
| | 28 | | 0.074 |
| Example 10 | 26.86 | +++ | |
| | 26.11 | | — |
| Example 11 | 24.87 | − | — |
| | 20.38 | | |
| Example 12 | 26.74 | − | — |
| | 26.04 | | |
| Example 13 | 26.01 | − | 0.245 |
| Example 14 | 19.82 | − | 0.377 |
| Example 15 | 27.92 | | |
| | 30.04 | | |
| Example 16 | — | − | 0.07 |
| Example 17 | — | − | 0.918 |
| Example 18 | 27.39 (bench | − | — |
| | control 27.08) | | |
| Example 19* | 23.72, 24.03 | − | — |
| Example 20* | 30.35, 30.56 | − | — |
| *Positive Control for Examples 19-20 was DNA extracted from two hundred (200) μL of whole blood following “Blood and Body Fluid Spin Protocol” described in QIAamp DNA Blood Mini Kit Handbook p. 27, eluting in 200 μL of water and had Ct value of 23. | | | |
| # Negative Control (NTC or no template control) did not amplify in these experiments. | | | |

The complete disclosures of the patents, patent documents, and publications cited herein are incorporated by reference in their entirety as if each were individually incorporated. Various modifications and alterations to this invention will become apparent to those skilled in the art without departing from the scope of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A method of isolating nucleic acid from a sample, the method comprising:
providing a sample comprising nucleic acid-containing material and inhibitors;
forming a concentrated region of the sample; wherein the concentrated region of the sample comprises nucleic acid-containing material and inhibitors;
substantially separating the concentrated region from a less concentrated region of the sample;
contacting the separated concentrated region of the sample with a solid phase material to preferentially adhere at least a portion of the inhibitors to the solid phase material, wherein the solid phase material comprises a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof;
optionally lysing the nucleic acid-containing material to release nucleic acid before, simultaneous with, or after contacting the separated concentrated region of the sample with the solid phase material; and
separating at least a portion of the nucleic acid-containing material and/or nucleic acid from the solid phase material having at least a portion of the inhibitors adhered thereto.

2. A method of isolating nucleic acid from a sample, the method comprising:
providing a sample comprising nucleic acid-containing material and cells containing inhibitors;
contacting the sample with a first lysing reagent under conditions effective to break cell membranes and release inhibitors and form a lysed sample comprising nucleic acid-containing material and inhibitors;
forming a concentrated region of the lysed sample; wherein the concentrated region of the lysed sample comprises nucleic acid-containing material and inhibitors;
substantially separating the concentrated region from a less concentrated region of the lysed sample;
contacting the separated concentrated region of the lysed sample with a solid phase material to preferentially adhere at least a portion of the inhibitors to the solid phase material, wherein the solid phase material comprises a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof;
optionally further lysing the nucleic acid-containing material to release nucleic acid before, simultaneous with, or after contacting the separated concentrated region of the sample with the solid phase material; and
separating at least a portion of the nucleic acid-containing material and/or nucleic acid from the solid phase material having at least a portion of the inhibitors adhered thereto.

3. A method of isolating nucleic acid from a sample, the method comprising:
providing a sample comprising cells containing inhibitors and cells containing nuclei;
contacting the biological sample with a nonionic surfactant under conditions effective to break cell membranes and release nuclei and inhibitors and form a lysed sample;
forming a concentrated region of the lysed sample; wherein the concentrated region of the sample comprises nuclei and inhibitors;
substantially separating the concentrated region of the lysed sample from the less concentrated region of the sample;
contacting the separated concentrated region of the lysed sample with a solid phase material to preferentially adhere at least a portion of the inhibitors to the solid phase material, wherein the solid phase material comprises a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof;
optionally further lysing the nuclei to release nucleic acid before, simultaneous with, or after contacting the separated concentrated region of the sample with the solid phase material; and
separating at least a portion of the nuclei and/or nucleic acid from the solid phase material having at least a portion of the inhibitors adhered thereto.

4. A method of isolating nucleic acid from a sample, the method comprising:
providing a microfluidic device comprising a loading chamber, a valved process chamber, and a separation chamber comprising a solid phase material;
providing a sample comprising nucleic acid-containing material and inhibitors;
placing the sample in the loading chamber;
transferring the sample to a valved process chamber;
forming a concentrated region of the sample in the valved process chamber, wherein the concentrated region of the sample comprises nucleic acid-containing material and inhibitors;
substantially separating the concentrated region from a less concentrated region of the sample;
transferring the separated concentrated region of the sample to the separation chamber for contact with the solid phase material to preferentially adhere at least a portion of the inhibitors to the solid phase material; wherein the solid phase material comprises capture sites, a coating reagent coated on the solid phase material, or both; wherein the coating reagent is selected from the group consisting of a surfactant, a strong base, a polyelectrolyte, a selectively permeable polymeric barrier, and combinations thereof;
optionally lysing the nucleic acid-containing material to release nucleic acid before, simultaneous with, or after contacting the separated concentrated region of the sample with the solid phase material; and
separating at least a portion of the nucleic acid-containing material and/or nucleic acid from the solid phase material having at least a portion of the inhibitors adhered thereto.

5. A method of isolating nucleic acid from a sample, the method comprising:
providing a microfluidic device comprising a loading chamber, a valved process chamber, and a separation chamber comprising a solid phase material;
providing a sample comprising nucleic acid-containing material and cells containing inhibitors;
placing the sample in the loading chamber;
contacting the sample with a first lysing reagent under conditions effective to break cell membranes and release inhibitors and form a lysed sample comprising nucleic acid-containing material and inhibitors;
transferring the lysed sample to a valved process chamber;
forming a concentrated region of the lysed sample in the valved process chamber, wherein the concentrated region of the lysed sample comprises nucleic acid-containing material and inhibitors;
substantially separating the concentrated region from a less concentrated region of the lysed sample;
transferring the separated concentrated region of the lysed sample to the separation chamber for contact with the solid phase material to preferentially adhere at least a portion of the inhibitors to the solid phase material; wherein the solid phase material comprises capture sites, a coating reagent coated on the solid phase material, or both; wherein the coating reagent is selected from the group consisting of a surfactant, a strong base, a polyelectrolyte, a selectively permeable polymeric barrier, and combinations thereof;
optionally further lysing the nucleic acid-containing material to release nucleic acid before, simultaneous with, or after contacting the separated concentrated region of the sample with the solid phase material; and
separating at least a portion of the nucleic acid-containing material and/or nucleic acid from the solid phase material having at least a portion of the inhibitors adhered thereto.

6. The method of claim 5 wherein the nucleic acid-containing material comprises nuclei and the method comprises separating at least a portion of the nuclei from the solid phase material.

7. The method of claim 5 wherein separating at least a portion of the nucleic acid-containing material and/or nucleic acid from the solid phase material comprises transferring the nucleic acid-containing material and/or nucleic acid to an amplification reaction chamber.

8. The method of claim 7 further comprising subjecting the nuclei and/or nucleic acid to an amplification reaction.

9. The method of claim 5 wherein separating at least a portion of the nucleic acid-containing material and/or nucleic acid from the solid phase material comprises eluting at least a portion of the nucleic acid-containing material and/or nucleic acid from the solid phase material with an eluting reagent, optionally heating the nucleic acid-containing material and/or nuclei, and optionally adjusting the pH.

10. A method of isolating nucleic acid from a sample, the method comprising:
providing a microfluidic device comprising a loading chamber, a valved process chamber, and a separation chamber comprising a solid phase material;
providing a sample comprising cells containing inhibitors and cells containing nuclei;
placing the sample in the loading chamber;
contacting the sample with a nonionic surfactant under conditions effective to break cell membranes and release nuclei and inhibitors to form a lysed sample;
transferring the lysed sample to a valved process chamber;
forming a concentrated region of the lysed sample in the valved process chamber, wherein the concentrated region of the lysed sample comprises nuclei and inhibitors;
substantially separating the concentrated region from a less concentrated region of the lysed sample;
transferring the separated concentrated region of the lysed sample to the separation chamber for contact with the solid phase material to preferentially adhere at least a portion of the inhibitors to the solid phase material; wherein the solid phase material comprises capture sites, a coating reagent coated on the solid phase material, or both; wherein the coating reagent is selected from the group consisting of a surfactant, a strong base, a polyelectrolyte, a selectively permeable polymeric barrier, and combinations thereof;
optionally further lysing the nuclei to release nucleic acid before, simultaneous with, or after contacting the separated concentrated region of the sample with the solid phase material; and
separating at least a portion of the nuclei and/or nucleic acid from the solid phase material having at least a portion of the inhibitors adhered thereto.

11. The method of claim 10 wherein separating at least a portion of the nuclei and/or nucleic acid from the solid phase material comprises transferring the nuclei and/or nucleic acid to an amplification reaction chamber.

12. The method of claim 11 further comprising optionally heating the nuclei and/or nucleic acid, optionally adjusting the pH, and subjecting the nuclei and/or nucleic acid to an amplification reaction.

13. The method of claim 10 wherein placing the sample in the loading chamber occurs prior to contacting the sample with a nonionic surfactant.

14. The method of claim 10 wherein the loading chamber comprises pre-deposited nonionic surfactant and contacting the sample with a nonionic surfactant occurs upon placing the sample in the loading chamber.

15. The method of claim 10 wherein placing the sample in the loading chamber occurs after contacting the sample with a nonionic surfactant.

16. The method of claim 10 wherein contacting the sample with a nonionic surfactant occurs in a mixing chamber with sufficient mixing to break cell membranes and release nuclei and inhibitors to form a lysed sample.

17. The method of claim 10 wherein forming a concentrated region of the sample in the valved process chamber comprises centrifuging the sample in the valved process chamber.

18. The method of claim 17 wherein separating the concentrated region from a less concentrated region of the sample comprises transferring the less concentrated region of the sample through the valve to a waste bin.

19. The method of claim 10 wherein the coating reagent comprises a surfactant.

20. The method of claim 10 wherein separating at least a portion of the nuclei and/or nucleic acid from the solid phase material comprises eluting at least a portion of the nuclei and/or nucleic acid from the solid phase material with an eluting reagent.

21. The method of claim 20 wherein the eluting reagent is a lysing reagent.

22. The method of claim 10 wherein the further lysing step is carried out with a second lysing reagent.

23. A method of isolating nucleic acid from a sample, the method comprising:
providing a sample comprising nucleic acid and inhibitors;
contacting the sample with a solid phase material to preferentially adhere at least a portion of the inhibitors to the solid phase material, wherein the solid phase material comprises a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof;
optionally lysing nucleic acid-containing material, if present, to release nucleic acid before, simultaneous with, or after contacting the lysed sample with the solid phase material; and
separating at least a portion of the nucleic acid-containing material and/or nucleic acid from the solid phase material having at least a portion of the inhibitors adhered thereto by eluting with an eluting reagent, with the proviso that the eluting reagent is not a nonionic surfactant.

24. A method of isolating nucleic acid from a sample, the method comprising:
providing a sample comprising nucleic acid-containing material and cells containing inhibitors;
contacting the sample with a first lysing reagent under conditions effective to break cell membranes and release inhibitors and form a lysed sample comprising nucleic acid-containing material and inhibitors;
contacting the lysed sample with a solid phase material to preferentially adhere at least a portion of the inhibitors to the solid phase material, wherein the solid phase material comprises a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof;
optionally further lysing the nucleic acid-containing material to release nucleic acid before, simultaneous with, or after contacting the lysed sample with the solid phase material; and
separating at least a portion of the nucleic acid-containing material and/or nucleic acid from the solid phase material having at least a portion of the inhibitors adhered thereto by eluting with an eluting reagent, with the proviso that the eluting reagent is not a nonionic surfactant.

25. A method of isolating nucleic acid from a sample, the method comprising:
providing a sample comprising nucleic acid-containing material and cells containing inhibitors;
contacting the sample with a first lysing reagent under conditions effective to break cell membranes and release inhibitors and form a lysed sample comprising nucleic acid-containing material and inhibitors;
contacting the lysed sample with a solid phase material to preferentially adhere at least a portion of the inhibitors to the solid phase material, wherein the solid phase material comprises a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof;
separating at least a portion of the nucleic acid-containing material from the solid phase material having at least a portion of the inhibitors adhered thereto; and
after separating the nucleic acid-containing material, further lysing the nucleic acid-containing material to release nucleic acid.

26. The method of claim 25 wherein the nucleic acid-containing material comprises nuclei.

27. The method of claim 25 wherein the coating reagent comprises a surfactant.

28. A method of isolating nucleic acid from a sample, the method comprising:
providing a microfluidic device comprising a loading chamber and a separation chamber comprising a solid phase material;
providing a sample comprising nucleic acid-containing material and cells containing inhibitors;
placing the sample in the loading chamber;
contacting the sample with a first lysing reagent under conditions effective to break cell membranes and release inhibitors and form a lysed sample comprising nucleic acid-containing material and inhibitors;
transferring the lysed sample to the separation chamber to contact the solid phase material and to preferentially adhere at least a portion of the inhibitors to the solid phase material;
optionally further lysing the nucleic acid-containing material to release nucleic acid before, simultaneous with, or after contacting the sample with the solid phase material; wherein the solid phase material comprises capture sites, a coating reagent coated on the solid phase material, or both; wherein the coating reagent is selected from the group consisting of a surfactant, a strong base, a polyelectrolyte, a selectively permeable polymeric barrier, and combinations thereof; and
separating at least a portion of the nucleic acid-containing material and/or nucleic acid from the solid phase material having at least a portion of the inhibitors adhered thereto by eluting with an eluting reagent, with the proviso that the eluting reagent is not a nonionic surfactant.

29. A method of isolating nucleic acid from a sample, the method comprising:
providing a microfluidic device comprising a loading chamber and a separation chamber comprising a solid phase material;
providing a sample comprising nucleic acid-containing material and cells containing inhibitors;
placing the sample in the loading chamber;
contacting the sample with a first lysing reagent under conditions effective to break cell membranes and release inhibitors and form a lysed sample comprising nucleic acid-containing material and inhibitors;
transferring the lysed sample to the separation chamber to contact the solid phase material and to preferentially adhere at least a portion of the inhibitors to the solid phase material; wherein the solid phase material comprises capture sites, a coating reagent coated on the solid phase material, or both; wherein the coating reagent is selected from the group consisting of a surfactant, a strong base, a polyelectrolyte, a selectively permeable polymeric barrier, and combinations thereof; and
separating at least a portion of the nucleic acid-containing material from the solid phase material having at least a portion of the inhibitors adhered thereto; and
after separating the nucleic acid-containing material, further lysing the nucleic acid-containing material to release nucleic acid.

30. The method of claim 29 wherein the nucleic acid-containing material comprises nuclei.

31. The method of claim 29 wherein separating the nucleic acid-containing material and/or nucleic acid from the solid phase material comprises transferring the nucleic acid-containing material and/or nucleic acid to an amplification reaction chamber.

32. The method of claim 31 further comprising subjecting the nucleic acid-containing material and/or nucleic acid to an amplification reaction.

33. The method of claim 29 wherein placing the sample in the loading chamber occurs prior to contacting the sample with a first lysing reagent.

34. The method of claim 29 wherein the loading chamber comprises the first lysing reagent and contacting the sample with a first lysing reagent occurs upon placing the sample in the loading chamber.

35. The method of claim 29 wherein placing the sample in the loading chamber occurs after contacting the sample with a first lysing reagent.

36. The method of claim 29 wherein forming a concentrated region of the sample in the valved process chamber comprises centrifuging the sample in the process chamber.

37. The method of claim 29 wherein the solid phase material comprises a polytetrafluoroethylene fibril matrix, sorptive particles enmeshed in the matrix, and a coating reagent coated on the solid phase material, wherein the coating reagent is selected from the group consisting of a surfactant, a strong base, a polyelectrolyte a selectively permeable polymeric barrier, and combinations thereof.

38. The method of claim 29 wherein the first lysing reagent is a nonionic surfactant.

39. The method of claim 29 wherein separating comprises eluting at least a portion of the nucleic acid-containing material from the solid phase material with an eluting reagent.

40. A kit for isolating nucleic acid from a sample, the kit comprising:
a first lysing reagent;
a solid phase material comprising a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof; and
instructions for lysing a sample and separating at least a portion of the nucleic acid-containing material and/or nucleic acid from the solid phase material according to the method of claim 2.

41. A kit for isolating nucleic acid from a sample, the kit comprising:
a first lysing reagent;
a solid phase material comprising a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier, and combinations thereof; and
instructions for lysing a sample and separating at least a portion of the nucleic acid-containing material and/or nucleic acid from the solid phase material according to the method of claim 24.

42. A kit for isolating nucleic acid from a sample, the kit comprising:
a nonionic surfactant;
a solid phase material comprising a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier and combinations thereof; and
instructions for lysing a sample and separating at least a portion of the nucleic acid-containing material and/or nucleic acid from the solid phase material according to the method of claim 3.

43. A kit for isolating nucleic acid from a sample, the kit comprising:
a nonionic surfactant;
a solid phase material comprising a polytetrafluoroethylene fibril matrix, sorptive particles enmeshed in the matrix, and a coating reagent coated on the solid phase material; wherein the coating reagent is selected from the group consisting of a surfactant, a strong base, a polyelectrolyte, a selectively permeable polymeric barrier and combinations thereof; and
instructions for lysing a sample and separating at least a portion of the nucleic acid-containing material and/or nucleic acid from the solid phase material according to the method of claim 25.

44. A kit for isolating nucleic acid from a sample, the kit comprising:
a first lysing reagent;
a microfluidic device comprising a loading chamber, a valved process chamber, and a separation chamber comprising a solid phase material; wherein the solid phase material comprises capture sites, a coating reagent coated on the solid phase material, or both; wherein the coating reagent is selected from the group consisting of a surfactant, a strong base, a polyelectrolyte, a selectively permeable polymeric barrier and combinations thereof; and
instructions for lysing a sample and separating at least a portion of the nucleic acid-containing material and/or nucleic acid from the solid phase material according to the method of claim 5.

45. A kit for isolating nucleic acid from a sample, the kit comprising:
a first lysing reagent;
a microfluidic device comprising a loading chamber, a valved process chamber, and a separation chamber comprising a solid phase material; wherein the solid phase material comprises capture sites, a coating reagent coated on the solid phase material, or both; wherein the coating reagent is selected from the group consisting of a surfactant, a strong base, a polyelectrolyte, a selectively permeable polymeric barrier and combinations thereof; and
instructions for lysing a sample and separating at least a portion of the nucleic acid-containing material and/or nucleic acid from the solid phase material according to the method of claim 28.

46. A kit for isolating nucleic acid from a sample, the kit comprising:
a nonionic surfactant;
a microfluidic device comprising a loading chamber, a valved process chamber, and a separation chamber comprising a solid phase material; wherein the solid phase material comprises capture sites, a coating reagent coated on the solid phase material, or both; wherein the coating reagent is selected from the group consisting of a surfactant, a strong base, a polyelectrolyte, a selectively permeable polymeric barrier and combinations thereof; and
instructions for lysing a sample and separating at least a portion of the nucleic acid-containing material and/or nucleic acid from the solid phase material according to the method of claim 10.

47. A kit for isolating nucleic acid from a sample, the kit comprising:
a nonionic surfactant;
a microfluidic device comprising a loading chamber, a valved process chamber, and a separation chamber comprising a solid phase material; wherein the solid phase material comprises capture sites, a coating reagent coated on the solid phase material, or both; wherein the coating reagent is selected from the group consisting of a surfactant, a strong base, a polyelectrolyte, a selectively permeable polymeric barrier and combinations thereof; and
instructions for lysing a sample and separating at least a portion of the nucleic acid-containing material and/or nucleic acid from the solid phase material according to the method of claim 29.
